SEQUENCE LISTING (85 sequences)

SEQ ID NO: 1 - DNA, 72 bp, synthetic construct
  source 1..72: mol_type = other DNA
cagcctcgggcagtgttgccttttacgaaatgttggtacagtgagtaccaatatgaggaccattcgctctca

SEQ ID NO: 2 - DNA, 68 bp, synthetic construct
  source 1..68: mol_type = other DNA
cagcctcgggcagtgttgccttttacgaaatgcaggtacagttggtacctgtctccacctcgctctcg

SEQ ID NO: 3 - DNA, 16 bp, synthetic construct
  source 1..16: mol_type = other DNA
tgacagcgcacgatca

SEQ ID NO: 4 - DNA, 21 bp, synthetic construct
  source 1..21: mol_type = other DNA
cagtgttgccttttacgaaat

SEQ ID NO: 5 - DNA, 67 bp, synthetic construct
  source 1..67: mol_type = other DNA
gagtacagtgccatgagagacttacgaaatgcaggtacagttggtacctgtctccaccagctggacg

SEQ ID NO: 6 - DNA, 72 bp, synthetic construct
  source 1..72: mol_type = other DNA
gagtacagtgccatgagagacttacgaaatgttggtacagtgagtaccaatatgaggaccatcagctggaca

SEQ ID NO: 7 - DNA, 20 bp, synthetic construct
  source 1..20: mol_type = other DNA
gcaaatacacagaggaagcc

SEQ ID NO: 8 - DNA, 22 bp, synthetic construct
  source 1..22: mol_type = other DNA
tgccatgagagacttacgaaat

SEQ ID NO: 9 - DNA, 72 bp, synthetic construct
  source 1..72: mol_type = other DNA
ctccaactaccacaagtttatttacgaaatgttggtacagtgagtaccaatatgaggaccatctacgccacc

SEQ ID NO: 10 - DNA, 67 bp, synthetic construct
  source 1..67: mol_type = other DNA
ctccaactaccacaagtttatttacgaaatgcaggtacagttggtacctgtctccacctacgccacg

SEQ ID NO: 11 - DNA, 32 bp, synthetic construct
  source 1..32: mol_type = other DNA
ttggtacagtgagtaccaatatgaggaccatc

SEQ ID NO: 12 - DNA, 27 bp, synthetic construct
  source 1..27: mol_type = other DNA
caggtacagttggtacctgtctccacc

SEQ ID NO: 13 - DNA, 19 bp, synthetic construct
  source 1..19: mol_type = other DNA
cctgctgaaaatgactgaa

SEQ ID NO: 14 - DNA, 23 bp, synthetic construct
  source 1..23: mol_type = other DNA
ccaactaccacaagtttatttac

SEQ ID NO: 15 - DNA, 72 bp, synthetic construct; identical to SEQ ID NO: 9
  source 1..72: mol_type = other DNA

SEQ ID NO: 16 - DNA, 67 bp, synthetic construct
  source 1..67: mol_type = other DNA
ctccaactaccacaagtttatttacgaaatgcaggtacagttggtacctgtctccacctacgccaca

SEQ ID NO: 17 - DNA, 32 bp, synthetic construct; identical to SEQ ID NO: 11
  source 1..32: mol_type = other DNA

SEQ ID NO: 18 - DNA, 27 bp, synthetic construct
  source 1..27: mol_type = other DNA
caggtacagttggtacctgtctccacc

SEQ ID NO: 19 - DNA, 19 bp, synthetic construct
  source 1..19: mol_type = other DNA
cctgctgaaaatgactgaa

SEQ ID NO: 20 - DNA, 23 bp, synthetic construct
  source 1..23: mol_type = other DNA
ccaactaccacaagtttatttac

SEQ ID NO: 21 - DNA, 42 bp, synthetic construct
  source 1..42: mol_type = other DNA
ttacgaaatgttggtacagtgagtaccaatatgaggaccatc

SEQ ID NO: 22 - DNA, 37 bp, synthetic construct
  source 1..37: mol_type = other DNA
ttacgaaatgcaggtacagttggtacctgtctccacc

SEQ ID NO: 23 - DNA, 72 bp, synthetic construct; identical to SEQ ID NO: 9
  source 1..72: mol_type = other DNA

SEQ ID NO: 24 - DNA, 67 bp, synthetic construct; identical to SEQ ID NO: 16
  source 1..67: mol_type = other DNA

SEQ ID NO: 25 - DNA, 32 bp, synthetic construct; identical to SEQ ID NO: 11
  source 1..32: mol_type = other DNA

SEQ ID NO: 26 - DNA, 27 bp, synthetic construct
  source 1..27: mol_type = other DNA
caggtacagttggtacctgtctccacc

SEQ ID NO: 27 - DNA, 19 bp, synthetic construct
  source 1..19: mol_type = other DNA
cctgctgaaaatgactgaa

SEQ ID NO: 28 - DNA, 23 bp, synthetic construct
  source 1..23: mol_type = other DNA
ccaactaccacaagtttatttac

SEQ ID NO: 29 - DNA, 72 bp, synthetic construct; identical to SEQ ID NO: 9
  source 1..72: mol_type = other DNA

SEQ ID NO: 30 - DNA, 67 bp, synthetic construct; identical to SEQ ID NO: 10
  source 1..67: mol_type = other DNA

SEQ ID NO: 31 - DNA, 32 bp, synthetic construct; identical to SEQ ID NO: 11
  source 1..32: mol_type = other DNA

SEQ ID NO: 32 - DNA, 27 bp, synthetic construct
  source 1..27: mol_type = other DNA
caggtacagttggtacctgtctccacc

SEQ ID NO: 33 - DNA, 19 bp, synthetic construct
  source 1..19: mol_type = other DNA
cctgctgaaaatgactgaa

SEQ ID NO: 34 - DNA, 23 bp, synthetic construct
  source 1..23: mol_type = other DNA
ccaactaccacaagtttatttac

SEQ ID NO: 35 - DNA, 72 bp, synthetic construct; identical to SEQ ID NO: 9
  source 1..72: mol_type = other DNA

SEQ ID NO: 36 - DNA, 67 bp, synthetic construct
  source 1..67: mol_type = other DNA
ctccaactaccacaagtttatttacgaaatgcaggtacagttggtacctgtctccacctacgccact

SEQ ID NO: 37 - DNA, 32 bp, synthetic construct; identical to SEQ ID NO: 11
  source 1..32: mol_type = other DNA

SEQ ID NO: 38 - DNA, 27 bp, synthetic construct
  source 1..27: mol_type = other DNA
caggtacagttggtacctgtctccacc

SEQ ID NO: 39 - DNA, 19 bp, synthetic construct
  source 1..19: mol_type = other DNA
cctgctgaaaatgactgaa

SEQ ID NO: 40 - DNA, 23 bp, synthetic construct
  source 1..23: mol_type = other DNA
ccaactaccacaagtttatttac

SEQ ID NO: 41 - DNA, 72 bp, synthetic construct; identical to SEQ ID NO: 9
  source 1..72: mol_type = other DNA

SEQ ID NO: 42 - DNA, 66 bp, synthetic construct
  source 1..66: mol_type = other DNA
ctccaactaccacaagtttatttacgaaatgcaggtacagttggtacctgtctccacctacgccaa

SEQ ID NO: 43 - DNA, 32 bp, synthetic construct; identical to SEQ ID NO: 11
  source 1..32: mol_type = other DNA

SEQ ID NO: 44 - DNA, 27 bp, synthetic construct
  source 1..27: mol_type = other DNA
caggtacagttggtacctgtctccacc

SEQ ID NO: 45 - DNA, 19 bp, synthetic construct
  source 1..19: mol_type = other DNA
cctgctgaaaatgactgaa

SEQ ID NO: 46 - DNA, 23 bp, synthetic construct
  source 1..23: mol_type = other DNA
ccaactaccacaagtttatttac

SEQ ID NO: 47 - DNA, 72 bp, synthetic construct; identical to SEQ ID NO: 9
  source 1..72: mol_type = other DNA

SEQ ID NO: 48 - DNA, 66 bp, synthetic construct
  source 1..66: mol_type = other DNA
ctccaactaccacaagtttatttacgaaatgcaggtacagttggtacctgtctccacctacgccag

SEQ ID NO: 49 - DNA, 32 bp, synthetic construct; identical to SEQ ID NO: 11
  source 1..32: mol_type = other DNA

SEQ ID NO: 50 - DNA, 27 bp, synthetic construct
  source 1..27: mol_type = other DNA
caggtacagttggtacctgtctccacc

SEQ ID NO: 51 - DNA, 19 bp, synthetic construct
  source 1..19: mol_type = other DNA
cctgctgaaaatgactgaa

SEQ ID NO: 52 - DNA, 23 bp, synthetic construct
  source 1..23: mol_type = other DNA
ccaactaccacaagtttatttac

SEQ ID NO: 53 - DNA, 74 bp, synthetic construct
  source 1..74: mol_type = other DNA
aatctcccctaaactccaattacgaaatgtactagcggcaagctagtgctagacttgacaccgctaaaacgacg

SEQ ID NO: 54 - DNA, 72 bp, synthetic construct
  source 1..72: mol_type = other DNA
aatctcccctaaactccaattacgaaatgcaggtacagttggtacctgtctccaccctccactaaaacaaca

SEQ ID NO: 55 - DNA, 27 bp, synthetic construct
  source 1..27: mol_type = other DNA
tactagcggcaagctagtgctagactt

SEQ ID NO: 56 - DNA, 27 bp, synthetic construct
  source 1..27: mol_type = other DNA
caggtacagttggtacctgtctccacc

SEQ ID NO: 57 - DNA, 23 bp, synthetic construct
  source 1..23: mol_type = other DNA
ccctaaactccaattacgaaatg

SEQ ID NO: 58 - DNA, 21 bp, synthetic construct
  source 1..21: mol_type = other DNA
cgcgggagattagaatttttg

SEQ ID NO: 59 - DNA, 22 bp, synthetic construct
  source 1..22: mol_type = other DNA
tgtgggagattagaatttttgg

SEQ ID NO: 60 - DNA, 80 bp, synthetic construct
  source 1..80: mol_type = other DNA
atcgcaaataaaaaccgaacatttccttacgaaatccaggtacagttggtacctgtctccacccaacgcacgataaaacg

SEQ ID NO: 61 - DNA, 83 bp, synthetic construct
  source 1..83: mol_type = other DNA
atcacaaataaaaaccaaacatttccttacgaaatctactagcggcaagctagtgctagacttcaacaacacacaataaaaca

SEQ ID NO: 62 - DNA, 27 bp, synthetic construct
  source 1..27: mol_type = other DNA
tactagcggcaagctagtgctagactt

SEQ ID NO: 63 - DNA, 27 bp, synthetic construct
  source 1..27: mol_type = other DNA
caggtacagttggtacctgtctccacc

SEQ ID NO: 64 - DNA, 22 bp, synthetic construct
  source 1..22: mol_type = other DNA
ccgaacatttccttacgaaatc

SEQ ID NO: 65 - DNA, 24 bp, synthetic construct
  source 1..24: mol_type = other DNA
aaccaaacatttccttacgaaatc

SEQ ID NO: 66 - DNA, 25 bp, synthetic construct
  source 1..25: mol_type = other DNA
gaaaagtgtaggttttataggtaga

SEQ ID NO: 67 - DNA, 74 bp, synthetic construct
  source 1..74: mol_type = other DNA
gaatacaggtattttgtccttgaagtattacgaaatgcaggtacagttggtacctgtctccacctggacaggca

SEQ ID NO: 68 - DNA, 78 bp, synthetic construct
  source 1..78: mol_type = other DNA
gaatacaggtattttgtccttgaagtattacgaaatgttggtacagtgagtaccaatatgaggaccatcggacaggcg

SEQ ID NO: 69 - DNA, 32 bp, synthetic construct; identical to SEQ ID NO: 11
  source 1..32: mol_type = other DNA

SEQ ID NO: 70 - DNA, 27 bp, synthetic construct
  source 1..27: mol_type = other DNA
caggtacagttggtacctgtctccacc

SEQ ID NO: 71 - DNA, 26 bp, synthetic construct
  source 1..26: mol_type = other DNA
aggtattttgtccttgaagtattacg

SEQ ID NO: 72 - DNA, 23 bp, synthetic construct
  source 1..23: mol_type = other DNA
ctaacatgttctagccagaagaa

SEQ ID NO: 73 - DNA, 16 bp, synthetic construct
  source 1..16: mol_type = other DNA
caccactaaaacaaca

SEQ ID NO: 74 - DNA, 23 bp, synthetic construct
  source 1..23: mol_type = other DNA
ccctaaactccaattacgaaatc

SEQ ID NO: 75 - DNA, 22 bp, synthetic construct
  source 1..22: mol_type = other DNA
ygygggagattagaatttttgg

SEQ ID NO: 76 - DNA, 19 bp, synthetic construct
  source 1..19: mol_type = other DNA
aatctcccctaaactccaa

SEQ ID NO: 77 - DNA, 13 bp, synthetic construct
  source 1..13: mol_type = other DNA
cgctaaaacgacg

SEQ ID NO: 78 - DNA, 20 bp, synthetic construct
  source 1..20: mol_type = other DNA
caacaacacacaataaaaca

SEQ ID NO: 79 - DNA, 17 bp, synthetic construct
  source 1..17: mol_type = other DNA
caacgcacgataaaacg

SEQ ID NO: 80 - DNA, 26 bp, synthetic construct
  source 1..26: mol_type = other DNA
atcacaaataaaaaccaaacatttcc

SEQ ID NO: 81 - DNA, 26 bp, synthetic construct
  source 1..26: mol_type = other DNA
atcgcaaataaaaaccgaacatttcc

SEQ ID NO: 82 - DNA, 24 bp, synthetic construct
  source 1..24: mol_type = other DNA
aaccraacatttccttacgaaatg

SEQ ID NO: 83 - DNA, 26 bp, synthetic construct
  source 1..26: mol_type = other DNA
atcacaaataaaaaccaaacatttcc

SEQ ID NO: 84 - DNA, 20 bp, synthetic construct
  source 1..20: mol_type = other DNA
caacaacacacaataaaaca

SEQ ID NO: 85 - DNA, 25 bp, synthetic construct
  source 1..25: mol_type = other DNA
gaaaagtgtaggttttataggtaga

(19) United States
(12) Patent Application Publication
(10) Pub. No.: US 2024/0368688 A1
(43) Pub. Date: Nov. 7, 2024

(54) METHODS AND COMPOSITIONS FOR DETECTION OF MUTANT NUCLEIC ACID SEQUENCES

(71) Applicant: TATAA Biocenter AB, Göteborg (SE)

(72) Inventors: Mikael Kubista, Göteborg (SE); Robert Sjöback, Göteborg (SE)

(21) Appl. No.: 18/594,668

(22) Filed: Mar. 4, 2024

Related U.S. Application Data
(63) Continuation of application No. PCT/EP2022/079299, filed on Oct. 20, 2022.
(60) Provisional application No. 63/257,954, filed on Oct. 20, 2021.

(51) Int. Cl. C12Q 1/6876; C12Q 1/6853

(52) U.S. Cl. CPC C12Q 1/6876; C12Q 1/6853; C12Q 2600/154

(57) ABSTRACT

Described herein are methods and compositions for detection of mutant nucleic acid sequences. In some cases, the methods and compositions used herein utilize a two-tailed primer in combination with one or more forward and reverse primers configured to hybridize to particular regions of the two-tailed primer to enable detection of a mutant sequence with high selectivity.

### CROSS-REFERENCE

This application is a continuation application of International Application No. PCT/EP2022/079299, filed on Oct. 20, 2022, which claims the benefit of U.S. Provisional Application No. 63/257,954, entitled “METHODS AND COMPOSITIONS FOR DETECTION OF MUTANT NUCLEIC ACID SEQUENCES”, filed on Oct. 20, 2021, which is incorporated by reference herein in its entirety.

### SEQUENCE LISTING

The instant application contains a Sequence Listing which has been submitted electronically in XML format and is hereby incorporated by reference in its entirety. Said XML copy, created on Feb. 28, 2024, is named 46005-702.301_SL.xml and is 77,364 bytes in size.

### BACKGROUND

Detection of mutant sequences using template-specific probe amplification in combination with quantitative methods such as e.g. quantitative polymerase chain reaction (qPCR), gel electrophoresis, or capillary electrophoresis is widely utilized patient genotyping for diagnostic and clinical purposes.

### SUMMARY

Existing schemes for detection of mutant sequences using template-specific probe amplification in combination with quantitative methods such as quantitative polymerase chain reaction (qPCR) suffer from lack of specificity for detection of mutant sequences, especially in the presence of wild-type sequences not containing the mutation. There is a need for design of new primer and probe compositions that improve selectivity of detection of mutant sequences in the presence of wild-type sequences.

Accordingly, in some embodiments, the current disclosure provides for methods, compositions, reaction mixtures, kits, and systems for processing a sequence variant to produce a detectable product with high selectivity. Such methods, compositions, reaction mixtures, kits, and systems can have utility in non-invasive prenatal testing (NIPT), cell-free deoxyribonucleic acid (DNA) analysis, patient genotyping (e.g. for tumor identification or autoimmune disease diagnosis), digital polymerase chain reaction (digital PCR), droplet digital PCR (ddPCR) Next-generation sequencing (NGS) sample prep, or detection of rejection after organ transplant (e.g. in the case of heart, lung, kidney, or liver transplant).

In some aspects, the present disclosure provides for a method for processing a DNA sequence having or suspected of having a sequence variant relative to a wild-type sequence, the method comprising: combining in a reaction mixture suitable for processing the DNA sequence: (i) the DNA sequence, wherein the DNA sequence comprises a variation of at least one nucleotide relative to the wild-type sequence; and (ii) a stem-loop primer that comprises: a 5′ hemiprobe sequence configured to hybridize to a complementary first end region of the DNA sequence; a stem-loop sequence; and a 3′ hemiprobe sequence configured to hybridize to a second end region of the DNA sequence, wherein a 3′ terminal portion of the 3′ hemiprobe sequence comprises a nucleotide complementary to the mismatch but not complementary to the wild-type sequence. In some embodiments, the method further comprises incubating the reaction mixture under conditions suitable to extend a product containing the 3′ hemiprobe sequence. In some embodiments, the method further comprises combining in the reaction mixture suitable for processing the product containing the 3′ hemiprobe sequence: a reverse primer configured to hybridize to a genomic region 3′ from the mismatch. In some embodiments, the reverse primer has a Tm of about 50-70 degrees Celsius. In some embodiments, the method further comprises incubating the reaction mixture suitable for processing the product containing the 3′ hemiprobe sequence under conditions suitable to produce extension products from reverse primer. In some embodiments, the method further comprises combining in a reaction mixture suitable for processing the product containing reverse primer sequence: the product containing the reverse primer sequence; and a forward primer configured to hybridize to: (i) at least part of the 5′ hemiprobe sequence; and (ii) at least part of a stem of the stem-loop sequence. In some embodiments, the forward primer comprises at least about 12 to about 30 nucleotides complementary to the 5′ hemiprobe sequence. In some embodiments, the forward primer comprises at least about 9 to about 35 nucleotides complementary to the stem of the stem-loop sequence 3′ to the nucleotides complementary to the 5′ hemiprobe sequence. In some embodiments, the forward primer has a Tm of about 50 to about 70 degrees Celsius. In some embodiments, the method further comprises incubating the reaction mixture suitable for processing the product containing reverse primer sequence under conditions suitable to produce extension products from the forward primer. In some embodiments, the reverse and the forward primer are in excess of or are at least about 20-fold, at least about 50-fold, at least about 100-fold, at least about 200-fold, at least about 500-fold, at least about 1,000-fold higher in concentration than a concentration of the two-tailed primer. In some embodiments, a concentration of the two-tailed primer is in excess of or is at least about 20-fold, at least about 50-fold, at least about 100-fold, at least about 200-fold, at least about 500-fold, at least about 1,000-fold higher in concentration than a concentration of the DNA sequence. In some embodiments, the stem-loop primer amplifies the mutant polynucleotide sequence at least about 10-fold, at least about 100-fold, at least about 1000-fold, at least about 10,000-fold, at least about 100,000-fold, or at least about 1,000,000-fold preferentially over the wild-type polynucleotide sequence. In some embodiments, the mutant polynucleotide sequence or wild-type polynucleotide sequence comprises genomic DNA. In some embodiments, the 5′ hemiprobe sequence comprises about 7 to about 22 nucleotides in length. In some embodiments, the 3′ hemiprobe sequence comprises about 3 to about 9 nucleotides in length. In some embodiments, the 3′ hemiprobe sequence has a Tm of about 30-40 degrees or the 5′ hemiprobe sequence has a Tm of about 60-75 degrees. In some embodiments, the stem-loop sequence comprises about 15 nucleotides in length or greater. In some embodiments, the stem-loop sequence is configured to have a Tm of about 55 to about 75 degrees Celsius. In some embodiments, a loop of the stem loop sequence comprises at least about 1 to at least about 20 nucleotides in length. In some embodiments, a loop of the stem loop sequence comprises a barcode. In some embodiments, the reaction mixture suitable for processing the product containing reverse primer sequence under conditions suitable to produce extension products from the forward primer further comprises an oligonucleotide probe comprising a detectable moiety, wherein the oligonucleotide probe is configured to hybridize to a complement of at least part of the stem-loop primer. In some embodiments, the at least part of the stem-loop primer comprises at least part of the stem-loop sequence. In some embodiments, the at least part of the stem-loop sequence comprises at least part of a loop sequence within the stem-loop sequence. In some embodiments, the detectable moiety comprises a 5′ fluorophore. In some embodiments, the oligonucleotide probe comprising the detectable moiety further comprises a quencher

In some aspects, the present disclosure provides for a kit for processing a DNA sequence, comprising: (a) a stem-loop primer that comprises: (i) a 5′ hemiprobe sequence configured to hybridize to a complementary first end region of the DNA sequence; (ii) a stem-loop sequence; and (iii) a 3′ hemiprobe sequence configured to hybridize to a second end region of the DNA sequence; (b) a forward primer configured to hybridize to: (i) at least part of the 5′ hemiprobe sequence; and (ii) at least part of a stem of the stem-loop sequence; and (c) a reverse primer configured to hybridize to a genomic region 3′ from the mismatch. In some embodiments, the DNA sequence has or is suspected of having a variation of at least one nucleotide relative to a wild-type sequence. In some embodiments, a 3′ terminal portion of the 3′ hemiprobe sequence comprises a nucleotide complementary to the variation but not complementary to the wild-type sequence. In some embodiments, the kit further comprises an oligonucleotide probe comprising a detectable moiety, wherein the oligonucleotide is configured to hybridize to at least part of the stem-loop primer. In some embodiments, the at least part of the stem-loop primer comprises at least part of the stem-loop sequence. In some embodiments, the at least part of the stem-loop sequence comprises at least part of a loop sequence within the stem-loop sequence. In some embodiments, the detectable moiety comprises a 5′ fluorophore. In some embodiments, the oligonucleotide probe comprising the detectable moiety further comprises a quencher. In some embodiments, the reverse primer has a Tm of about 50-70 degrees Celsius. In some embodiments, the forward primer comprises at least about 12 to about 30 nucleotides complementary to the 5′ hemiprobe sequence. In some embodiments, the forward primer comprises at least about 9 to about 35 nucleotides complementary to the stem of the stem-loop sequence 3′ to the nucleotides complementary to the 5′ hemiprobe sequence. In some embodiments, the forward primer has a Tm of about 50 to about 70 degrees Celsius. In some embodiments, the stem-loop primer amplifies the mutant polynucleotide sequence at least about 10-fold, at least about 100-fold, at least about 1000-fold, at least about 10,000-fold, at least about 100,000-fold, or at least about 1,000,000-fold preferentially over the wild-type polynucleotide sequence. In some embodiments, the 5′ hemiprobe sequence comprises about 7 to about 22 nucleotides in length. In some embodiments, the 3′ hemiprobe sequence comprises about 3 to about 9 nucleotides in length. In some embodiments, the 3′ hemiprobe sequence has a Tm of about 30-40 degrees or the 5′ hemiprobe sequence has a Tm of about 60-75 degrees. In some embodiments, the stem-loop sequence comprises about 15 nucleotides in length or greater. In some embodiments, the stem-loop sequence is configured to have a Tm of about 55 to about 75 degrees Celsius. In some embodiments, a loop of the stem loop sequence comprises at least about 1 to at least about 20 nucleotides in length. In some embodiments, a loop of the stem loop sequence comprises a barcode. In some embodiments, the stem-loop primer, the forward primer, or the reverse primer comprise any one of SEQ ID NOs: 1, 2, 3, 4, 5, 6, 7, 8, 9, 10, 13, 14, 15, 16, 19, 20, 23, 24, 27, 28, 29, 30, 33, 34, 35, 36, 39, 40, 41, 42, 45, 46, 47, 48, 51, 52, 53, 54, 57, 58, 59, 60, 61, 64, 65, 66, 67, 68, 71, or 72. In some aspects, the present disclosure provides for a composition for processing a DNA sequence, comprising: (a) a stem-loop primer that comprises: (i) a 5′ hemiprobe sequence configured to hybridize to a complementary first end region of the DNA sequence; (ii) a stem-loop sequence; and (iii) a 3′ hemiprobe sequence configured to hybridize to a second end region of the DNA sequence; (b) a forward primer configured to hybridize to: (i) at least part of the 5′ hemiprobe sequence; and (ii) at least part of a stem of the stem-loop sequence; and (c) a reverse primer configured to hybridize to a genomic region 3′ from the mismatch, wherein a concentration of the forward primer or a concentration of the reverse primer are at least 10-fold higher than a concentration of the stem-loop primer. In some embodiments, the DNA sequence has or is suspected of having a variation of at least one nucleotide relative to a wild-type sequence. In some embodiments, the 3′ hemiprobe sequence comprises a nucleotide complementary to the variation but not complementary to the wild-type sequence. In some embodiments, the composition further comprises an oligonucleotide probe comprising a detectable moiety, wherein the oligonucleotide is configured to hybridize to at least part of the stem-loop primer. In some embodiments, the at least part of the stem-loop primer comprises at least part of the stem-loop sequence. In some embodiments, the at least part of the stem-loop sequence comprises at least part of a loop sequence within the stem-loop sequence. In some embodiments, the detectable moiety comprises a 5′ fluorophore. In some embodiments, the oligonucleotide probe comprising the detectable moiety further comprises a quencher. In some embodiments, the concentration of the forward primer or the concentration of the reverse primer are in excess of or are at least about 20-fold, at least about 50-fold, at least about 100-fold, at least about 200-fold, at least about 500-fold, at least about 1,000-fold higher than the concentration of the stem-loop primer. In some embodiments, a 3′ terminal portion of the 3′ hemiprobe sequence comprises a nucleotide complementary to the mismatch but not complementary to the wild-type sequence. In some embodiments, the reverse primer has a Tm of about 50-70 degrees Celsius. In some embodiments, the forward primer comprises at least about 12 to about 30 nucleotides complementary to the 5′ hemiprobe sequence. In some embodiments, the forward primer comprises at least about 9 to about 35 nucleotides complementary to the stem of the stem-loop sequence 3′ to the nucleotides complementary to the 5′ hemiprobe sequence. In some embodiments, the forward primer has a Tm of about 50 to about 70 degrees Celsius. In some embodiments, the stem-loop primer amplifies the mutant polynucleotide sequence at least about 10-fold, at least about 100-fold, at least about 1000-fold, at least about 10,000-fold, at least about 100,000-fold, or at least about 1,000,000-fold preferentially over the wild-type polynucleotide sequence. In some embodiments, the 5′ hemiprobe sequence comprises about 7 to about 22 nucleotides in length. In some embodiments, the 3′ hemiprobe sequence comprises about 3 to about 9 nucleotides in length. In some embodiments, the 3′ hemiprobe sequence has a Tm of about 30-40 degrees or the 5′ hemiprobe sequence has a Tm of about 60-75 degrees. In some embodiments, the stem-loop sequence comprises about 15 nucleotides in length or greater. In some embodiments, the stem-loop sequence is configured to have a Tm of about 55 to about 75 degrees Celsius. In some embodiments, a loop of the stem loop sequence comprises at least about 1 to at least about 20 nucleotides in length. In some embodiments, a loop of the stem loop sequence comprises a barcode. In some embodiments, the forward primer, or the reverse primer comprise any one of SEQ ID NOs: 1, 2, 3, 4, 5, 6, 7, 8, 9, 10, 13, 14, 15, 16, 19, 20, 23, 24, 27, 28, 29, 30, 33, 34, 35, 36, 39, 40, 41, 42, 45, 46, 47, 48, 51, 52, 53, 54, 57, 58, 59, 60, 61, 64, 65, 66, 67, 68, 71, or 72. In some embodiments, the incubating comprises a PCR reaction, a qPCR reaction, a dPCR reaction, a ddPCR reaction, or a sequencing reaction.

In some aspects, the present disclosure provides for a method for processing a DNA sequence having or suspected of having a methylated cytosine at a particular residue, the method comprising: combining in a reaction mixture suitable for processing the DNA sequence: (i) the DNA sequence, wherein the DNA sequence has been treated with bisulfite and comprises a uracil at a cytosine residue that was non-methylated prior to the bisulfite treatment; and (ii) a first stem-loop primer that comprises: a 5′ hemiprobe sequence configured to hybridize to a complementary first end region of the DNA sequence; a stem-loop sequence; and a 3′ hemiprobe sequence configured to hybridize to a second end region of the DNA sequence, wherein a 3′ terminal portion of the 3′ hemiprobe sequence comprises a nucleotide complementary to the uracil but not complementary to the cytosine residue. In some embodiments, the method further comprises incubating the reaction mixture under conditions suitable to extend a product containing the 3′ hemiprobe sequence. In some embodiments, the method further comprises combining in the reaction mixture suitable for processing the product containing the 3′ hemiprobe sequence: a reverse primer configured to hybridize to a genomic region 3′ from the mismatch. In some embodiments, the reverse primer has a Tm of about 50-70 degrees Celsius. In some embodiments, the method further comprises incubating the reaction mixture suitable for processing the product containing the 3′ hemiprobe sequence under conditions suitable to produce extension products from reverse primer. In some embodiments, the method further comprises combining in a reaction mixture suitable for processing the product containing reverse primer sequence: the product containing the reverse primer sequence; and a forward primer configured to hybridize to: (i) at least part of the 5′ hemiprobe sequence; and (ii) at least part of a stem of the stem-loop sequence. In some embodiments, the forward primer comprises at least about 12 to about 30 nucleotides complementary to the 5′ hemiprobe sequence. In some embodiments, the forward primer comprises at least about 9 to about 35 nucleotides complementary to the stem of the stem-loop sequence 3′ to the nucleotides complementary to the 5′ hemiprobe sequence. In some embodiments, the forward primer has a Tm of about 50 to about 70 degrees Celsius. In some embodiments, the method further comprises incubating the reaction mixture suitable for processing the product containing reverse primer sequence under conditions suitable to produce extension products from the forward primer. In some embodiments, the method further comprises providing the DNA sequence. In some embodiments, the method further comprises treating the DNA sequence with bisulfite prior to the combining. In some embodiments, the incubating comprises a PCR reaction, a qPCR reaction, a dPCR reaction, a ddPCR reaction, or a sequencing reaction.

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

### INCORPORATION BY REFERENCE

All publications, patents, and patent applications mentioned in this specification are herein incorporated by reference to the same extent as if each individual publication, patent, or patent application was specifically and individually indicated to be incorporated by reference

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 (FIG. 1) depicts an example mutant-sensitive detection assay using stem-loop primers according to some of the embodiments of the disclosure. In this assay, the presence of a mutant residue in e.g. genomic DNA (highlighted) allows the extension of a 3′ hemiprobe region of a stem-loop primer in a first extension reaction. In a second phase of this assay, the extended stem-loop primer is combined with a reverse primer that binds to a genomic region 3′ of the 3′ hemiprobe in a second extension reaction, allowing production of a second strand corresponding to the extended stem-loop primer containing the reverse primer sequence. Finally, the product containing the reverse-primer sequence is combined in an 3^{rd} extension reaction with a forward primer spanning part of the 5′ hemiprobe and stem regions; inclusion of a probe binding a sequence 5′ of this forward primer optionally allows detection of this product by e.g. qPCR.

FIGS. 2A and 2B (FIGS. 2A and 2B) depict designed conditions (FIG. 2A) and qPCR traces (FIG. 2B) for an optimization experiment described in Example 1 for detecting an ACTN3 mutant. FIG. 2B bottom panel shows traces for amplification of sequences containing mutant ACTN3 with the ACTN3 mutant detecting stem loop primer; FIG. 2B top panel shows a chart of RFU for amplification from mutant detecting stem-loop primer for reactions containing homozygous WT, homozygous mutant, and heterozygote ACTN3 sequences, indicating that the 3 genotypes can be distinguished by PCR.

FIGS. 3A and 3B (FIGS. 3A and 3B) depict designed conditions (FIG. 3A) and qPCR traces (FIG. 3B) for an optimization experiment described in Example 1 for detecting an NRAS mutant. FIG. 3B bottom panel shows traces for amplification of sequences containing mutant NRAS with the NRAS mutant detecting stem loop primer; FIG. 3B top panel shows a chart of RFU for amplification from mutant detecting stem-loop primer for reactions containing homozygous WT, homozygous mutant, and heterozygote ACTN3 sequences, indicating that the 3 genotypes can be distinguished by PCR.

FIGS. 4A and 4B (FIGS. 4A and 4B) depict results for an experiment designed to assess selectivity of the mutant ACTN3 detecting and NRAS detecting stem-loop primers. FIG. 4A depicts reaction design for the selectivity assays. FIG. 4B top panel depicts Cq values for the FAM labelled (mutant) and HEX labelled (wild-type) probes, respectively, measured at different ratios of targets (mutant/WT) as described in (A) for the ACTN3 assay; FIG. 4B bottom panel depicts Cq values for the FAM labelled (mutant) and HEX labelled (wild-type) probes, respectively, measured for different ratios of targets as described in FIG. 4A for the NRAS assay.

FIGS. 5A, 5B, 5C, and 5D (FIGS. 5A, 5B, 5C, and 5D) depict examples of digital PCR data of SNP detecting stem-loop primer assays from two different experiments. FIGS. 5A, 5B, and 5C) depict results for an experiment designed to assess the sensitivity of G12R KRAS mutant detecting stem-loop primer assay on samples with different WT/mutant target template ratios (between 0.05% and 50% mutant to WT ratio) on the QIAcuity Digital PCR System. FIG. 5A depicts numerical data from the experiment; FIG. 5B depicts 1D amplitude plots and FIG. 5C depicts examples of 2D amplitude plots from the same experiment, demonstrating that very few dots corresponding to the proper category mis-segregate. FIG. 5D depicts results from a set of experiments designed to assess the function of a G12R KRAS mutant detecting stem-loop primer assay on different dPCR platforms. The results depicted are 2D amplitude plots and abbreviated numerical result tables from the same assay run on samples with 50% WT and 50% mutant target template on three different dPCR platforms: QX200 Droplet Digital PCR System, Naica System for Crystal Digital PCR and QIAcuity Digital PCR System

FIG. 6 (FIG. 6) depicts 2D amplification plots for the five different KRAS mutant detecting stem-loop assays all using same the generic stem-loop sequences and complementary probes as in Example 7.

FIG. 7 (FIG. 7) shows 2D amplitude plots for the experiment depicted in Table 14 and Example 7.

FIGS. 8A and 8B (FIGS. 8A and 8B) depict design of primers and methylation discrimination for the experiment described in Example 8 operating on CORO6 sequences. FIG. 8A shows a schematic of a methylation-detecting 2T-primer (CORO6-2T.M) designed to target the CORO6 gene, with hemiprobes in black text (bold/underlined) and stem loop sequence and arms in dark grey lines, the target sequence in black text, the extended 2T-primer sequence (in grey text), the reverse and forward primer sequence (in black italics). The probe (not shown) binds selectively to the complement of the stem loop sequence and arms of the 2T-primer. The target DNA has small letters on original cytosine-sites that via bisulfite-treatment may turn into uracils (represented in the figure and in synthetic DNA sequences as thymines), while methylated CpG-sites have grey highlight (which in non-methylated DNA can be represented by TG). FIG. 8B shows allelic discrimination performance of the 2T-assay using the components from FIG. 8A in qPCR on synthetic gBlock sequences representing methylated DNA, non-methylated DNA, mixed methylated/non-methylated DNA and a no template control (NTC).

FIGS. 9A and 9B (FIGS. 9A and 9B) depict design of primers and methylation discrimination for the experiment described in Example 9 operating on FAM101A sequences. FIG. 9A shows a schematic of a non-methylation-detecting 2T-primer for detecting methylation status of the FAM101A gene (FAM101A-2T.NM with hemiprobes in black text (bold/underlined) and stem loop sequence and arms in dark grey lines, the target sequence in black text, the reverse and forward primer sequence (in black italics). The probe (not shown) binds selectively to the complement of the stem loop sequence and arms of the 2T-primer. The target DNA has been modified so that original “single” cytosine-sites are represented by thymine (since bisulfite-treatment may turn such cytosines into uracils) while original CpG-sites have grey highlight (which in the non-methylated DNA template and in the figure are be represented by TG). FIG. 9B shows allelic discrimination performance of the 2T-assay in qPCR on synthetic gBlock sequences representing methylated DNA, non-methylated DNA, mixed methylated/non-methylated DNA and a no template control (NTC). At annealing temperatures of around 55-62° C., the assay performs robustly and a clear distinction of different type of template DNA is made while keeping false-positive signal very low and limited to the FAM-channel (detecting methylated DNA)

FIGS. 10A and 10B (FIGS. 10A and 10B) show results of biallelic discrimination of CORO6 and FAM101A methylation on common samples. FIG. 10A depicts qPCR allelic discrimination results when analysing of methylation/non-methylation representative gBlocks of genes CORO6 and FAM101A that were synthetically produced (M.gB—methylated target; NM.gB—non-methylated target; Mix.gB—50/50 mix of M/NM-gBlocks) alongside two unique bisulfite treated (BST) DNA samples extracted from white blood cells (WBCs) and heart tissue (40 ng DNA/reaction (before BST)). CORO6 and FAM101A primers were constructed as in previous examples, and qPCR to detect both markers was performed as in previous examples. HEX fluorophore is a signal for methylated CORO6 sequence and non-methylated FAM101A. As predicted by the documented behaviour of CORO6 and FAM101A, WBCs show signal in the FAM-channel, while heart show signal in both HEX- and FAM-channel, showing that both assays can detect heart DNA in a background of white blood cells (the main source of DNA in cfDNA). FIG. 10B depicts results when samples described in FIG. 10A were analysed with the FAM101A 2T-assay using digital PCR (QIAcuity, Qiagen) instead of qPCR. Heart samples show a mixed signal (FAM/HEX), while WBCs show signal for the methylated DNA (FAM). The NTC show a relatively high background fluorescence in the NTC, but the signal is limited to the FAM-channel, and as such detection of heart-specific signal (HEX) is not compromised.

FIG. 11 (FIG. 11) depicts results of the crude blood genotyping experiment of Example 11. The left panel of FIG. 11 shows duplicate qPCR measurement on homozygote wild type (top), homozygote mutant (middle) and heterozygote (bottom). The right panel shows a plot clustering the measured data based on fluorescence intensity clearly distinguishing the duplicate two homoduplexes and the heteroduplex. The plot in the right panel clearly demonstrates that wild-type, heterozygous, and mutant can be discriminated from whole crude blood without additional purification steps.

### DETAILED DESCRIPTION

### Definitions

The terminology used herein is for the purpose of describing particular cases only and is not intended to be limiting. As used herein, the singular forms “a” “an” and “the” are intended to include the plural forms as well, unless the context clearly indicates otherwise. Furthermore, to the extent that the terms “including”, “includes”, “having”, “has”, “with”, or variants thereof are used in either the detailed description and/or the claims, such terms are intended to be inclusive in a manner similar to the term “comprising.”

The term “about” or “approximately” generally refers to an amount that is near the stated amount by about 10%, 5%, or 1%, including increments therein. For example, “about” or “approximately” can mean a range including the particular value and ranging from 10% below that particular value and spanning to 10% above that particular value.

The practice of some methods disclosed herein employ, unless otherwise indicated, techniques of immunology, biochemistry, chemistry, molecular biology, microbiology, cell biology, genomics and recombinant DNA. See for example Sambrook and Green, Molecular Cloning: A Laboratory Manual, 4th Edition (2012); the series Current Protocols in Molecular Biology (F. M. Ausubel, et al. eds.); the series Methods In Enzymology (Academic Press, Inc.), PCR 2: A Practical Approach (M. J. MacPherson, B. D. Hames and G. R. Taylor eds. (1995)), Harlow and Lane, eds. (1988) Antibodies, A Laboratory Manual, and Culture of Animal Cells: A Manual of Basic Technique and Specialized Applications, 6th Edition (R. I. Freshney, ed. (2010)) (which is entirely incorporated by reference herein).

The terms “polynucleotide,” “nucleic acid,” and “oligonucleotide,” as used herein, generally refer to a polymeric form of nucleotides of any length, either deoxyribonucleotides or ribonucleotides, or analogs thereof. Polynucleotides may have any three-dimensional structure, and may perform any function, known or unknown. The following are non-limiting examples of polynucleotides: coding or non-coding regions of a gene or gene fragment, intergenic DNA, loci (locus) defined from linkage analysis, exons, introns, messenger RNA (mRNA), transfer RNA, ribosomal RNA, short interfering RNA (siRNA), short-hairpin RNA (shRNA), micro-RNA (miRNA), small nucleolar RNA, ribozymes, cDNA, recombinant polynucleotides, branched polynucleotides, plasmids, vectors, isolated DNA of any sequence, isolated RNA of any sequence, nucleic acid probes, adapters, and primers. A polynucleotide may comprise modified nucleotides, such as methylated nucleotides and nucleotide analogs. If present, modifications to the nucleotide structure may be imparted before or after assembly of the polymer. The sequence of nucleotides may be interrupted by non-nucleotide components. A polynucleotide may be further modified after polymerization, such as by conjugation with a labeling component, tag, reactive moiety, or binding partner. Polynucleotide sequences, when provided, are listed in the 5′ to 3′ direction, unless stated otherwise.

“Hybridizes,” and “annealing,”, as used herein, generally refer to a reaction in which one or more polynucleotides interact to form a complex that is stabilized via hydrogen bonding between the bases of the nucleotide residues. The hydrogen bonding may occur by Watson Crick base pairing, Hoogstein binding, or in any other sequence sensitive or specific manner. The complex may comprise two strands forming a duplex structure, three or more strands forming a multi stranded complex, a single self-hybridizing strand, or any combination of these. A hybridization reaction may constitute a step in a more extensive process, such as the initiation of a PCR, or the enzymatic cleavage of a polynucleotide by a ribozyme. A first sequence that can be stabilized via hydrogen bonding with the bases of the nucleotide residues of a second sequence can generally be said to be “hybridizable” to the second sequence. In such a case, the second sequence can also be said to be hybridizable to the first sequence.

“Complement,” “complements,” “complementary,” and “complementarity,”, as used herein, generally refer to a sequence that is fully complementary to and hybridizable to the given sequence. In some cases, a first sequence that is hybridizable to a second sequence or set of second sequences is specifically or selectively hybridizable to the second sequence or set of second sequences, such that hybridization to the second sequence or set of second sequences is used. Hybridizable sequences can share a degree of sequence complementarity over all or a portion of their respective lengths, such as between 25%-100% complementarity, including at least about 25%, 30%, 35%, 40%, 45%, 50%, 55%, 60%, 65%, 70%, 75%, 80%, 85%, 90%, 91%, 92%, 93%, 94%, 95%, 96%, 97%, 98%, 99%, and 100% sequence complementarity.

As used herein, the term “homology” generally refers to a nucleotide sequence which is homologous to a reference nucleotide sequence. Degree of homology and complementarity can vary in accordance with a given application, and can be more than 5%, 10%, 20%, 30%, 40%, 50%, 60%, 70%, 80%, 90%, or more than 95%.

As used herein, the terms “amplify,” “amplifies,” “amplified,” “amplification,” and “amplicon” generally refer to any method for replicating a nucleic acid with the use of a primer-dependent polymerase and/or those processes' products. In some cases, the amplification is effected by PCR using a pair of primers, comprising a first and second primer as described above. Amplified products can be subjected to subsequence analyses, including but not limited to melting curve analysis, nucleotide sequencing, single-strand conformation polymorphism assay, allele-specific oligonucleotide hybridization, Southern blot analysis, and restriction endonuclease digestion.

Amplification products may be detected by the use of a probe. As used herein, the term “probe” generally refers to a polynucleotide that carries a detectable member and has complementarity to a target nucleic acid, thus being able to hybridize with said target and be detected by said detectable member. In certain embodiments, a probe may include Watson-Crick bases or modified bases. Modified bases include, but are not limited to, the AEGIS bases described, e.g., in U.S. Pat. Nos. 5,432,272; 5,965,364; and 6,001,983, each of which are entirely incorporated by reference herein. In certain aspects, bases are joined by a natural phosphodiester bond or a different chemical linkage. Different chemical linkages include, but are not limited to, a peptide bond, an LNA linkage, or a phosphorothioate linkage.

Amplification can be performed by any suitable method. The nucleic acids may be amplified by polymerase chain reaction (PCR), as described in, for example, U.S. Pat. Nos. 5,928,907 and 6,015,674, each of which are incorporated by reference herein for any purpose. Other methods of nucleic acid amplification may include, for example, ligase chain reaction, oligonucleotide ligations assay, and hybridization assay, as described in greater detail in U.S. Pat. Nos. 5,928,907 and 6,015,674, each of which are incorporated by reference herein in their entirety. Methods can involve real-time optical detection systems described in greater detail in, for example, U.S. Pat. Nos. 5,928,907 and 6,015,674, each which are incorporated by reference herein. Other amplification methods that can be used according to some methods of the disclosure include those described in U.S. Pat. Nos. 5,242,794; 5,494,810; 4,988,617; and 6,582,938, each of which are incorporated herein in their entirety.

### Example Embodiments

In some aspects, the present disclosure provides for a method for processing a nucleic acid sequence. In some cases, the sequence has or is suspected of having a sequence variation relative to a wild-type sequence. In some cases, the nucleic acid sequence comprises DNA. The nucleic acid sequence can comprise essentially any type of sequence. In some cases, the sequence having or suspected of having a mutation comprises double-stranded DNA, such as genomic DNA. In some cases, the sequence having or suspected of having a mutation comprises a gene region, such as an open-reading frame, an exon, an intron, or a splice junction. In some cases, the sequence having or suspected of having a mutation comprises an intergenic region, such as a promoter, enhancer, or insulator region. In some cases, the sequence having or suspected of having a mutation comprises a region of a particular gene, such as a region of a RAS gene (e.g. KRAS, NRAS, HRAS) or a region of a ACTN3 gene.

In some embodiments, the method comprises: combining in a reaction mixture suitable for processing the nucleic acid sequence: (i) the nucleic acid sequence, wherein the nucleic acid sequence comprises a mismatch of at least one nucleotide relative to the wild-type sequence; and (ii) a stem-loop primer. In some embodiments, processing comprises amplifying, and involves the addition of accessory enzymes (e.g. polymerases), dNTPs, buffers, or chemical stabilizers (e.g. DMSO, DTT, mannitol, betaine) necessary to perform an amplification reaction. In some embodiments, the stem-loop primer comprises: a 5′ hemiprobe sequence configured to hybridize to a complementary first end region of the nucleic acid sequence; a stem-loop sequence; and a 3′ hemiprobe sequence. In some embodiments, the 3′ hemiprobe sequence is configured to hybridize to a second end region of the nucleic acid sequence. In some embodiments a 3′ terminal portion of the 3′ hemiprobe sequence comprises a nucleotide complementary to the mismatch but not to complementary to the wild-type sequence. In some embodiments, the method further comprises incubating the reaction mixture under conditions suitable to extend a product containing the 3′ hemiprobe sequence. In some embodiments, the method further comprises combining in the reaction mixture suitable for processing the product containing the 3′ hemiprobe sequence: a reverse primer configured to hybridize to a genomic region 3′ from the mismatch. In some embodiments, in said reaction mixture, said reverse and said forward primer are in excess of or are at least about 20-fold, at least about 50-fold, at least about 100-fold, at least about 200-fold, at least about 500-fold, at least about 1,000-fold higher in concentration than a concentration of said two-tailed primer. In some embodiments, in said reaction mixture, a concentration of said two-tailed primer is in excess of or is at least about 20-fold, at least about 50-fold, at least about 100-fold, at least about 200-fold, at least about 500-fold, at least about 1,000-fold higher in concentration than a concentration of said DNA sequence. In some embodiments, the reverse primer has a Tm of at least about 50, 52, 54, 56, 58, 60, 62, 64, 66, or 68 degrees Celsius. In some embodiments, the reverse primer has a Tm of at most about 52, 54, 56, 58, 60, 62, 64, 66, 68, or 70 degrees Celsius. In some embodiments, the reverse primer has a Tm of about 50 to about 70 degrees Celsius. In some embodiments, the method further comprises incubating the reaction mixture suitable for processing the product containing the 3′ hemiprobe sequence under conditions suitable to produce extension products from reverse primer. In some embodiments, the method further comprises combining in a reaction mixture suitable for processing the product containing reverse primer sequence: the product containing the reverse primer sequence; and a forward primer configured to hybridize to: (i) at least part of the 5′ hemiprobe sequence; or (ii) at least part of a stem of the stem-loop sequence. In some embodiments, the forward primer comprises at least about 12, 13, 14, 15, 16, 17, 18, 19, 20, 21, 22, 23, 24, 25, 26, 27, 28, or 29 nucleotides to at most about 13, 14, 15, 16, 17, 18, 19, 20, 21, 22, 23, 24, 25, 26, 27, 28, 29, or 30 nucleotides complementary to the 5′ hemiprobe sequence. In some embodiments, the forward primer comprises at least about 9, 10, 11, 12, 13, 14, 15, 16, 17, 18, 19, 20, 21, 22, 23, 24, 25, 26, 27, 28, 29, 30, 31, 32, 33, or 34 to at most about 10, 11, 12, 13, 14, 15, 16, 17, 18, 19, 20, 21, 22, 23, 24, 25, 26, 27, 28, 29, 30, 31, 32, 33, 34, or 35 nucleotides complementary to the stem of the stem-loop sequence 3′ to the nucleotides complementary to the 5′ hemiprobe sequence. In some embodiments, the forward primer has a Tm at least about 50, 52, 54, 56, 58, 60, 62, 64, 66, or 68 degrees Celsius. In some embodiments, the forward primer has a Tm of at most about 52, 54, 56, 58, 60, 62, 64, 66, 68, or 70 degrees Celsius. In some embodiments, the forward primer has a Tm of about 50 to about 70 degrees Celsius. In some embodiments, the method further comprises incubating the reaction mixture suitable for processing the product containing reverse primer sequence under conditions suitable to produce extension products from the forward primer. In some embodiments, the stem-loop primer amplifies the mutant polynucleotide sequence at least about 10-fold, at least about 100-fold, at least about 1000-fold, at least about 10,000-fold, at least about 100,000-fold, or at least about 1,000,000-fold preferentially over the wild-type polynucleotide sequence. In some embodiments, the mutant polynucleotide sequence or wild-type polynucleotide sequence comprises genomic DNA. In some embodiments, the 5′ hemiprobe sequence comprises at least about 7, 8, 9, 10, 11, 12, 13, 14, 15, 16, 17, 18, 19, 20, 21, or 22 to at most about 8, 9, 10, 11, 12, 13, 14, 15, 16, 17, 18, 19, 20, 21, or 22 nucleotides in length. In some embodiments, the 3′ hemiprobe sequence comprises at least about 3, 4, 5, 6, 7, or 8 nucleotides to at most about 4, 5, 6, 7, 8, or 9 nucleotides in length. In some embodiments, the 3′ hemiprobe sequence has a Tm of at least about 30, 31, 32, 33, 34, 35, 36, 37, 38, or 39 degrees Celsius to at most about 31, 32, 33, 34, 35, 36, 37, 38, 39, or 40 degrees Celsius. In some embodiments, the 5′ hemiprobe sequence has a Tm of at least about 60, 61, 62, 63, 64, 65, 66, 67, 68, 69, 70, 71, 72, 73, or 74 degrees Celsius to at most about 61, 62, 63, 64, 65, 66, 67, 68, 69, 70, 71, 72, 73, 74, or 75 degrees Celsius. In some embodiments, the stem-loop sequence comprises about 5, 8, 10, 12, or 15 nucleotides in length or greater. In some embodiments, the stem-loop sequence is configured to have a Tm of at least about 55, 57, 59, 61, 63, 65, 67, 69, 71, or 72 degrees Celsius to at most about 57, 59, 61, 63, 65, 67, 69, 71, or 75 degrees Celsius. In some embodiments, a loop of the stem loop sequence comprises at least about 1 to at least about 20 nucleotides in length. In some embodiments, a loop of the stem loop sequence comprises a barcode. In some embodiments, said reaction mixture suitable for processing said product containing reverse primer sequence under conditions suitable to produce extension products from said forward primer further comprises an oligonucleotide probe comprising a detectable moiety, wherein said oligonucleotide probe is configured to hybridize to a complement of at least part of said-stem-loop primer. In some embodiments, said oligonucleotide probe comprises a sequence homologous to at least part of said stem-loop primer. In some embodiments, said at least part of said stem-loop primer comprises at least part of said stem-loop sequence. In some embodiments, said at least part of said stem-loop sequence comprises at least part of a loop sequence within said stem-loop sequence. In some embodiments, said detectable moiety comprises a fluorophore. In some embodiments, the fluorophore is a 5′-fluorophore. In some embodiments, said oligonucleotide probe comprising said detectable moiety further comprises a quencher. In some embodiments, said quencher is a 3′ quencher. In some embodiments, said quencher is an internal quenches (e.g. attached to an internal residue or nucleotide of said oligonucleotide probe). In some embodiments, said stem-loop sequence comprises a mismatch in a stem of said stem-loop sequence. In some embodiment, said stem-loop sequence comprises at least 1, at least 2, at least 3, at least 4, at least 5, or at least 6 mismatches in a stem of said stem-loop sequence. In some cases, a stem of said stem loop is configured to have a Tm of between 50 and 70 degrees Celsius. In some cases, said stem loop comprises at least about 40 to at least about 70 nucleotides. In some embodiments, the stem-loop primer, the forward primer, or the reverse primer comprise any one of SEQ ID NOs: 1, 2, 3, 4, 5, 6, 7, 8, 9, 10, 13, 14, 15, 16, 19, 20, 23, 24, 27, 28, 29, 30, 33, 34, 35, 36, 39, 40, 41, 42, 45, 46, 47, 48, 51, 52, 53, 54, 57, 58, 59, 60, 61, 64, 65, 66, 67, 68, 71, or 72 In some cases, said method comprises combining in said reaction mixture suitable for processing the nucleic acid sequence a plurality of different stem-loop primers comprising 5′ or 3′ regions with specificity for different DNA sequences. In some cases, in the use of a plurality of a plurality of different stem-loop primers comprising 5′ or 3′ regions with specificity for different DNA sequences, such composition can enable the detection of multiple different DNA sequences without unique molecular identifiers or UMIs (e.g. by detection of varying lengths of stem-loops incorporated within the stem-loop primers).

In some aspects, the present disclosure provides for a kit for processing a nucleic acid sequence having or suspected of having a mutation relative to a wild-type sequence. In some embodiments, the kit comprises: (a) a stem-loop primer that comprises: (i) a 5′ hemiprobe sequence configured to hybridize to a complementary first end region of the nucleic acid sequence; (ii) a stem-loop sequence; and (iii) a 3′ hemiprobe sequence configured to hybridize to a second end region of the nucleic acid sequence, wherein a 3′ terminal portion of the 3′ hemiprobe sequence comprises a nucleotide complementary to the mismatch but not to complementary to the wild-type sequence; (b) a forward primer configured to hybridize to: (i) at least part of the 5′ hemiprobe sequence; and (ii) at least part of a stem of the stem-loop sequence; and (c) a reverse primer configured to hybridize to a genomic region 3′ from the mismatch. In some embodiments, the kit further comprises an oligonucleotide probe comprising a detectable moiety, wherein said oligonucleotide is configured to hybridize to at least part of said stem-loop primer. In some embodiments, said at least part of said stem-loop primer comprises at least part of said stem-loop sequence. In some embodiments, said at least part of said stem-loop sequence comprises at least part of a loop sequence within said stem-loop sequence. In some embodiments, said detectable moiety comprises a 5′ fluorophore. In some embodiments, said oligonucleotide probe comprising said detectable moiety further comprises a quencher. In some embodiments, said quencher is a 3′ quencher. In some embodiments, said quencher is an internal quencher (e.g. linked to an internal residue or nucleotide of said oligonucleotide probe). In some embodiments, said stem-loop sequence comprises a mismatch in a stem of said stem-loop sequence. In some embodiment, said stem-loop sequence comprises at least 1, at least 2, at least 3, at least 4, at least 5, or at least 6 mismatches in a stem of said stem-loop sequence. In some cases, a stem of said stem loop is configured to have a Tm of at least about 50 to at least about 70 degrees Celsius. In some cases, said stem loop comprises at least about 40 to at least about 70 nucleotides. In some cases, said kit comprises a plurality of different stem-loop primers comprising 5′ or 3′ regions with specificity for different DNA sequences. In some cases, in the use of a plurality of a plurality of different stem-loop primers comprising 5′ or 3′ regions with specificity for different DNA sequences, such composition can enable the detection of multiple different DNA sequences without unique molecular identifiers or UMIs (e.g. by detection of varying lengths of stem-loops incorporated within the stem-loop primers). In some embodiments, the reverse primer has a Tm of about 50-70 degrees Celsius. In some embodiments, the forward primer comprises at least about 12 to about 30 nucleotides complementary to the 5′ hemiprobe sequence. In some embodiments, the forward primer comprises at least about 9 to about 35 nucleotides complementary to the stem of the stem-loop sequence 3′ to the nucleotides complementary to the 5′ hemiprobe sequence. In some embodiments, the forward primer has a Tm of about 50 to about 70 degrees Celsius. In some embodiments, the stem-loop primer amplifies the mutant polynucleotide sequence at least about 10-fold, at least about 100-fold, at least about 1000-fold, at least about 10,000-fold, at least about 100,000-fold, or at least about 1,000,000-fold preferentially over the wild-type polynucleotide sequence. In some embodiments, the 5′ hemiprobe sequence comprises about 7 to about 22 nucleotides in length. In some embodiments, the 3′ hemiprobe sequence comprises about 3 to about 9 nucleotides in length. In some embodiments, the 3′ hemiprobe sequence has a Tm of about 30-40 degrees or the 5′ hemiprobe sequence has a Tm of about 60-75 degrees. In some embodiments, the stem-loop sequence comprises about 15 nucleotides in length or greater. In some embodiments, the stem-loop sequence is configured to have a Tm of about 55 to about 75 degrees Celsius. In some embodiments, a loop of the stem loop sequence comprises at least about 1 to at least about 20 nucleotides in length. In some embodiments, a loop of the stem loop sequence comprises a barcode. In some embodiments, the stem-loop primer, the forward primer, or the reverse primer comprise any one of SEQ ID NOs: 1, 2, 3, 4, 5, 6, 7, 8, 9, 10, 13, 14, 15, 16, 19, 20, 23, 24, 27, 28, 29, 30, 33, 34, 35, 36, 39, 40, 41, 42, 45, 46, 47, 48, 51, 52, 53, 54, 57, 58, 59, 60, 61, 64, 65, 66, 67, 68, 71, or 72.

In some aspects, the present disclosure provides for a composition for processing a nucleic acid sequence having or suspected of having a mutation relative to a wild-type sequence, comprising: (a) a stem-loop primer that comprises: (i) a 5′ hemiprobe sequence configured to hybridize to a complementary first end region of the nucleic acid sequence; (ii) a stem-loop sequence; and (iii) a 3′ hemiprobe sequence configured to hybridize to a second end region of the nucleic acid sequence; (b) a forward primer configured to hybridize to: (i) at least part of the 5′ hemiprobe sequence; and (ii) at least part of a stem of the stem-loop sequence; and (c) a reverse primer configured to hybridize to a genomic region 3′ from the mismatch, wherein a concentration of the forward primer or a concentration of the reverse primer are at least 10-fold higher than a concentration of the stem-loop primer. In some embodiments, said composition further comprises an oligonucleotide probe comprising a detectable moiety, wherein said oligonucleotide is configured to hybridize to at least part of said stem-loop primer. In some embodiments, said at least part of said stem-loop primer comprises at least part of said stem-loop sequence. In some embodiments, said at least part of said stem-loop sequence comprises at least part of a loop sequence within said stem-loop sequence. In some embodiments, said detectable moiety comprises a 5′ fluorophore. In some embodiments, said oligonucleotide probe comprising said detectable moiety further comprises a quencher. In some embodiments, said quencher is a 3′ quencher. In some embodiments, said quencher is an internal quencher (e.g. linked to an internal residue or nucleotide of said oligonucleotide probe). In some embodiments, said stem-loop sequence comprises a mismatch in a stem of said stem-loop sequence. In some embodiment, said stem-loop sequence comprises at least 1, at least 2, at least 3, at least 4, at least 5, or at least 6 mismatches in a stem of said stem-loop sequence. In some cases, a stem of said stem loop is configured to have a Tm of between 50 and 70 degrees Celsius. In some cases, said stem loop comprises at least about 40 to at least about 70 nucleotides. In some cases, said composition comprises a plurality of different stem-loop primers comprising 5′ or 3′ regions with specificity for different DNA sequences. In some cases, in the use of a plurality of a plurality of different stem-loop primers comprising 5′ or 3′ regions with specificity for different DNA sequences, such composition can enable the detection of multiple different DNA sequences without unique molecular identifiers or UMIs (e.g. by detection of varying lengths of stem-loops incorporated within the stem-loop primers). In some embodiments, the concentration of the forward primer or the concentration of the reverse primer are at least about 20-fold, at least about 50-fold, at least about 100-fold, at least about 200-fold, at least about 500-fold, at least about 1,000-fold higher than the concentration of the stem-loop primer. In some embodiments, a 3′ terminal portion of the 3′ hemiprobe sequence comprises a nucleotide complementary to the mismatch but not to complementary to the wild-type sequence. In some embodiments, the reverse primer has a Tm of about 50-70 degrees Celsius. In some embodiments, the forward primer comprises at least about 12 to about 30 nucleotides complementary to the 5′ hemiprobe sequence. In some embodiments, the forward primer comprises at least about 9 to about 35 nucleotides complementary to the stem of the stem-loop sequence 3′ to the nucleotides complementary to the 5′ hemiprobe sequence. In some embodiments, the forward primer has a Tm of about 50 to about 70 degrees Celsius. In some embodiments, the stem-loop primer amplifies the mutant polynucleotide sequence at least about 10-fold, at least about 100-fold, at least about 1000-fold, at least about 10,000-fold, at least about 100,000-fold, or at least about 1,000,000-fold preferentially over the wild-type polynucleotide sequence. In some embodiments, the 5′ hemiprobe sequence comprises about 7 to about 22 nucleotides in length. In some embodiments, the 3′ hemiprobe sequence comprises about 3 to about 9 nucleotides in length. In some embodiments, the 3′ hemiprobe sequence has a Tm of about 30-40 degrees or the 5′ hemiprobe sequence has a Tm of about 60-75 degrees. In some embodiments, the stem-loop sequence comprises about 15 nucleotides in length or greater. In some embodiments, the stem-loop sequence is configured to have a Tm of about 55 to about 75 degrees Celsius. In some embodiments, a loop of the stem loop sequence comprises at least about 1 to at least about 20 nucleotides in length. In some embodiments, a loop of the stem loop sequence comprises a barcode. In some embodiments, the stem-loop primer, the forward primer, or the reverse primer comprise any one of SEQ ID NOs: 1, 2, 3, 4, 5, 6, 7, 8, 9, 10, 13, 14, 15, 16, 19, 20, 23, 24, 27, 28, 29, 30, 33, 34, 35, 36, 39, 40, 41, 42, 45, 46, 47, 48, 51, 52, 53, 54, 57, 58, 59, 60, 61, 64, 65, 66, 67, 68, 71, or 728 or any of the sequences described in Table 1.

TABLE 1
Sequences of Primer or Oligonucleotide Components Described Herein

| SEQ | | |
|---|---|---|
| ID | | |
| NO: | NAME | SEQUENCE (DNA) |
| 1 | 2T- | CAGCCTCGGGCAGTGTTGCCTTTTACGAAATGTTGGTACAGTGAGTACCA |
| | ACTN3_Mut_05-2 | ATATGAGGACCATTCGCTCTCA |
| 2 | 2T- | CAGCCTCGGGCAGTGTTGCCTTTTACGAAATGCAGGTACAGTTGGTACCT |
| | ACTN3_WT_05-2 | GTCTCCACCTCGCTCTCG |
| 3 | 2T_ACTN3_Rv_03 | TGACAGCGCACGATCA |
| 4 | 2T_ACTN3_Fw_03 | CAGTGTTGCCTTTTACGAAAT |
| 5 | 2T-NRAS_Mut_01 | GAGTACAGTGCCATGAGAGACTTACGAAATGCAGGTACAGTTGGTACCTG |
| | | TCTCCACCAGCTGGACG |
| 6 | 2T-NRAS_WT_01 | GAGTACAGTGCCATGAGAGACTTACGAAATGTTGGTACAGTGAGTACCAA |
| | | TATGAGGACCATCAGCTGGACA |
| 7 | 2T_NRAS_Rv_01 | GCAAATACACAGAGGAAGCC |
| 8 | 2T_NRAS_Fw_01 | TGCCATGAGAGACTTACGAAAT |
| 9 | 2T WT primer | CTCCAACTACCACAAGTTTATTTACGAAATGTTGGTACAGTGAGTACCAA |
| | (example 3) | TATGAGGACCATCTACGCCACC |
| 10 | 2T Mut primer | CTCCAACTACCACAAGTTTATTTACGAAATGCAGGTACAGTTGGTACCTG |
| | (example 3) | TCTCCACCTACGCCACG |
| 11 | WT probe (example | TTGGTACAGTGAGTACCAATATGAGGACCATC |
| | 3) | |
| 12 | Mut probe (example | CAGGTACAGTTGGTACCTGTCTCCACC |
| | 3) | |
| 13 | Forward primer | CCTGCTGAAAATGACTGAA |
| | (example 3) | |
| 14 | Reverse primer | CCAACTACCACAAGTTTATTTAC |
| | (example 3) | |
| 15 | 2T WT primer | CTCCAACTACCACAAGTTTATTTACGAAATGTTGGTACAGTGAGTACCAA |
| | (example 4) | TATGAGGACCATCTACGCCACC |
| 16 | 2T Mut primer | CTCCAACTACCACAAGTTTATTTACGAAATGCAGGTACAGTTGGTACCTG |
| | (KRAS 29T) | TCTCCACCTACGCCACA |
| | (example 4) | |
| 17 | WT probe (example | TTGGTACAGTGAGTACCAATATGAGGACCATC |
| | 4) | |
| 18 | Mut probe (KRAS | CAGGTACAGTTGGTACCTGTCTCCACC |
| | 29T) (example 4) | |
| 19 | Forward primer | CCTGCTGAAAATGACTGAA |
| | (example 4) | |
| 20 | Reverse primer | CCAACTACCACAAGTTTATTTAC |
| | (example 4) | |
| 21 | Generic WT | TTACGAAATGTTGGTACAGTGAGTACCAATATGAGGACCATC |
| | detection stem-loop | |
| | sequence (example | |
| | 5) | |
| 22 | Generic mutant | TTACGAAATGCAGGTACAGTTGGTACCTGTCTCCACC |
| | detection stem-loop | |
| | sequence (example | |
| | 5) | |
| 23 | 2T WT primer | CTCCAACTACCACAAGTTTATTTACGAAATGTTGGTACAGTGAGTACCAA |
| | KRAS 29T | TATGAGGACCATCTACGCCACC |
| 24 | 2T Mut primer | CTCCAACTACCACAAGTTTATTTACGAAATGCAGGTACAGTTGGTACCTG |
| | KRAS 29T | TCTCCACCTACGCCACA |
| 25 | WT probe KRAS | TTGGTACAGTGAGTACCAATATGAGGACCATC |
| | 29T | |
| 26 | Mut probe KRAS | CAGGTACAGTTGGTACCTGTCTCCACC |
| | 29T | |
| 27 | Forward primer | CCTGCTGAAAATGACTGAA |
| | KRAS 29T | |
| 28 | Reverse primer | CCAACTACCACAAGTTTATTTAC |
| | KRAS 29T | |
| 29 | 2T WT primer | CTCCAACTACCACAAGTTTATTTACGAAATGTTGGTACAGTGAGTACCAA |
| | KRAS 29C | TATGAGGACCATCTACGCCACC |
| 30 | 2T Mut primer | CTCCAACTACCACAAGTTTATTTACGAAATGCAGGTACAGTTGGTACCTG |
| | KRAS 29C | TCTCCACCTACGCCACG |
| 31 | WT probe KRAS | TTGGTACAGTGAGTACCAATATGAGGACCATC |
| | 29C | |
| 32 | Mut probe KRAS | CAGGTACAGTTGGTACCTGTCTCCACC |
| | 29C | |
| 33 | Forward primer | CCTGCTGAAAATGACTGAA |
| | KRAS 29C | |
| 34 | Reverse primer | CCAACTACCACAAGTTTATTTAC |
| | KRAS 29C | |
| 35 | 2T WT primer | CTCCAACTACCACAAGTTTATTTACGAAATGTTGGTACAGTGAGTACCAA |
| | KRAS 29A | TATGAGGACCATCTACGCCACC |
| 36 | 2T Mut primer | CTCCAACTACCACAAGTTTATTTACGAAATGCAGGTACAGTTGGTACCTG |
| | KRAS 29A | TCTCCACCTACGCCACT |
| 37 | WT probe KRAS | TTGGTACAGTGAGTACCAATATGAGGACCATC |
| | 29A | |
| 38 | Mut probe KRAS | CAGGTACAGTTGGTACCTGTCTCCACC |
| | 29A | |
| 39 | Forward primer | CCTGCTGAAAATGACTGAA |
| | KRAS 29A | |
| 40 | Reverse primer | CCAACTACCACAAGTTTATTTAC |
| | KRAS 29A | |
| 41 | 2T WT primer | CTCCAACTACCACAAGTTTATTTACGAAATGTTGGTACAGTGAGTACCAA |
| | KRAS 30T | TATGAGGACCATCTACGCCACC |
| 42 | 2T Mut primer | CTCCAACTACCACAAGTTTATTTACGAAATGCAGGTACAGTTGGTACCTG |
| | KRAS 30T | TCTCCACCTACGCCAA |
| 43 | WT probe KRAS | TTGGTACAGTGAGTACCAATATGAGGACCATC |
| | 30T | |
| 44 | Mut probe KRAS | CAGGTACAGTTGGTACCTGTCTCCACC |
| | 30T | |
| 45 | Forward primer | CCTGCTGAAAATGACTGAA |
| | KRAS 30T | |
| 46 | Reverse primer | CCAACTACCACAAGTTTATTTAC |
| | KRAS 30T | |
| 47 | 2T WT primer | CTCCAACTACCACAAGTTTATTTACGAAATGTTGGTACAGTGAGTACCAA |
| | (KRAS 30C) | TATGAGGACCATCTACGCCACC |
| 48 | 2T Mut primer | CTCCAACTACCACAAGTTTATTTACGAAATGCAGGTACAGTTGGTACCTG |
| | (KRAS 30C) | TCTCCACCTACGCCAG |
| 49 | WT probe (KRAS | TTGGTACAGTGAGTACCAATATGAGGACCATC |
| | 30C) | |
| 50 | Mut probe (KRAS | CAGGTACAGTTGGTACCTGTCTCCACC |
| | 30C) | |
| 51 | Forward primer | CCTGCTGAAAATGACTGAA |
| | (KRAS 30C) | |
| 52 | Reverse primer | CCAACTACCACAAGTTTATTTAC |
| | (KRAS 30C) | |
| 53 | CORO6.2T-M | AATCTCCCCTAAACTCCAATTACGAAATGTACTAGCGGCAAGCTAGTGCT |
| | | AGACTTGACACCGCTAAAACGACG |
| 54 | CORO6.2T-NM | AATCTCCCCTAAACTCCAATTACGAAATGCAGGTACAGTTGGTACCTGTC |
| | | TCCACCCTCCACTAAAACAACA |
| 55 | 2T-Universal HEX | TACTAGCGGCAAGCTAGTGCTAGACTT |
| | (methylation) probe | |
| 56 | 2T-Universal FAM | CAGGTACAGTTGGTACCTGTCTCCACC |
| | (non-methylation) | |
| | probe | |
| 57 | CORO6.F | CCCTAAACTCCAATTACGAAATG |
| 58 | CORO6.R-M | CGCGGGAGATTAGAATTTTTG |
| 59 | CORO6.R-NM | TGTGGGAGATTAGAATTTTTGG |
| 60 | FAM101A.2T-M | ATCGCAAATAAAAACCGAACATTTCCTTACGAAATCCAGGTACAGTTGGT |
| | | ACCTGTCTCCACCCAACGCACGATAAAACG |
| 61 | FAM101A.2T-NM | ATCACAAATAAAAACCAAACATTTCCTTACGAAATCTACTAGCGGCAAGC |
| | | TAGTGCTAGACTTCAACAACACACAATAAAACA |
| 62 | 2T-Universal HEX | TACTAGCGGCAAGCTAGTGCTAGACTT |
| | (non-methylation) | |
| | probe | |
| 63 | 2T-Universal FAM | CAGGTACAGTTGGTACCTGTCTCCACC |
| | (methylation) probe | |
| 64 | FAM101A.F-M | CCGAACATTTCCTTACGAAATC |
| 65 | FAM101A.F-NM | AACCAAACATTTCCTTACGAAATC |
| 66 | FAM101A.R | GAAAAGTGTAGGTTTTATAGGTAGA |
| 67 | Factor V | GAATACAGGTATTTTGTCCTTGAAGTATTACGAAATGCAGGTACAGTTGG |
| | (“Leiden”) | TACCTGTCTCCACCTGGACAGGCA |
| | mutation 2T-primer | |
| 68 | Factor V WT 2T- | GAATACAGGTATTTTGTCCTTGAAGTATTACGAAATGTTGGTACAGTGAG |
| | primer | TACCAATATGAGGACCATCGGACAGGCG |
| 69 | WT factor V probe | TTGGTACAGTGAGTACCAATATGAGGACCATC |
| 70 | Mutant (leiden | CAGGTACAGTTGGTACCTGTCTCCACC |
| | mutation) factor V | |
| | probe | |
| 71 | Forward primer | AGGTATTTTGTCCTTGAAGTATTACG |
| | factor V | |
| 72 | Reverse primer | CTAACATGTTCTAGCCAGAAGAA |
| | factor V | |

In some cases, an RT reaction and/or DNA amplification reaction (e.g. PCR) can be carried out in droplets, such as in droplet digital PCR. The droplets used herein can include emulsion compositions (or mixtures of two or more immiscible fluids) as described in U.S. Pat. No. 7,622,280. The droplets can be generated by devices described in WO/2010/036352. The term emulsion, as used herein, can refer to a mixture of immiscible liquids (such as oil and water). Oil-phase and/or water-in-oil emulsions allow for the compartmentalization of reaction mixtures within aqueous droplets. The emulsions can comprise aqueous droplets within a continuous oil phase. The emulsions provided herein can be oil-in-water emulsions, wherein the droplets can be oil droplets within a continuous aqueous phase. In some cases, the droplets are configured to prevent mixing between compartments, with each compartment protecting its contents from evaporation and coalescing with the contents of other compartments.

Splitting a sample into small reaction volumes (e.g. for droplet digital PCR) can enable the use of reduced amounts of reagents, thereby lowering the material cost of the analysis. Reducing sample complexity by partitioning also improves the dynamic range of detection because higher-abundance molecules are separated from low-abundance molecules in different compartments, thereby allowing lower-abundance molecules greater proportional access to reaction reagents, which in turn enhances the detection of lower-abundance molecules.

Droplets can be generated having an average diameter of about, less than, at least, or more than 0.001, 0.01, 0.05, 0.1, 1, 5, 10, 20, 30, 40, 50, 60, 70, 80, 100, 120, 130, 140, 150, 160, 180, 200, 300, 400, or 500 microns. Droplets can have an average diameter of about 0.001 to about 500, about 0.01 to about 500, about 0.1 to about 500, about 0.1 to about 100, about 0.01 to about 100, or about 1 to about 100 microns. Microfluidic methods of producing emulsion droplets using microchannel cross-flow focusing or physical agitation can produce either monodisperse or polydisperse emulsions. The droplets can be monodisperse droplets. The droplets can be generated such that the size of the droplets does not vary by more than plus or minus 5% of the average size of the droplets. In some cases, the droplets can be generated such that the size of the droplets does not vary by more than plus or minus 2% of the average size of the droplets. A droplet generator can generate a population of droplets from a single sample, wherein none of the droplets vary in size by more than plus or minus about 0.1%, 0.5%>, 1%>, 1.5%, 2%, 2.5%, 3%, 3.5%, 4%, 4.5%, 5%, 5.5%, 6%, 6.5%, 7%, 7.5%, 8%, 8.5%, 9%, 9.5%, or 10% of the average size of the total population of droplets.

A droplet can be formed by flowing an oil phase through an aqueous sample. The aqueous phase can comprise a buffered solution and reagents for performing a PCR reaction, including nucleotides, primers, probe(s) for fluorescent detection, template nucleic acids, DNA polymerase enzyme, and optionally, reverse transcriptase enzyme.

The aqueous phase can comprise a buffered solution and reagents for performing a PCR reaction without solid-state beads, such as magnetic-beads.

A non-specific blocking agent such as BSA or gelatin from bovine skin can be used in the aqueous phase, wherein the gelatin or BSA is present in a concentration range of about 0.1 to about 0.9% w/v. Other possible blocking agents can include betalactoglobulin, casein, dry milk, or other common blocking agents. In some cases, concentrations of BSA and gelatin are about 0.1% w/v.

In some cases, the aqueous phase can also comprise additives including, but not limited to, non-specific background/blocking nucleic acids (e.g., salmon sperm DNA), biopreservatives (e.g. sodium azide), PCR enhancers (e.g. Betaine, Trehalose, etc.), and inhibitors (e.g. RNAse inhibitors).

In some cases, a non-ionic Ethylene Oxide/Propylene Oxide block copolymer is added to the aqueous phase in a concentration of about 0.1%, 0.2%, 0.3%, 0.4%, 0.5%, 0.6%, 0.7%, 0.8%), 0.9%), or 1.0%). Common biosurfactants can include non-ionic surfactants such as Pluronic F-68, Tetronics, Zonyl FSN.

The oil phase can comprise a fluorinated base oil which can be additionally stabilized by combination with a fluorinated surfactant such as a perfluorinated polyether. In some cases, the base oil can be one or more of HFE 7500, FC-40, FC-43, FC-70, or other common fluorinated oil.

The oil phase can further comprise an additive for tuning the oil properties, such as vapor pressure or viscosity or surface tension. Nonlimiting examples include perfluoro-octanol and 1H,1H,2H,2H-Perfluorodecanol.

In some cases, droplets of the emulsion can be generated using commercially available droplet generator, such as Bio-Rad QX100™ Droplet Generator. RT and the droplet PCR can be carried out using commercially available, and the droplet is analyzed using commercially available droplet reader such as generator, such as Bio-Rad QX100™ Droplet Reader.

In some cases, the amplifying step is carried out by performing digital PCR, such as microfluidic-based digital PCR or droplet digital PCR.

In some cases, the digital PCR is performed in droplets having a volume that is between about 1 pL and about 100 nL.

In some cases, droplet generation can comprise introducing encapsulating dyes, such as fluorescent molecules, in droplets, for example, with a known concentration of dyes, where the droplets are suspended in an immiscible carrier fluid, such as oil, to form an emulsion.

Example fluorescent dyes that can used with any methods according to the current disclosure include a fluorescein derivative, such as carboxyfluorescein (FAM), and a PULSAR 650 dye (a derivative of Ru(bpy)3). FAM has a relatively small Stokes shift, while Pulsar® 650 dye has a very large Stokes shift. Both FAM and PULSAR 650 dye can be excited with light of approximately 460-480 nm. FAM emits light with a maximum of about 520 nm (and not substantially at 650 nm), while PULSAR 650 dye emits light with a maximum of about 650 nm (and not substantially at 520 nm).

Carboxyfluorescein can be paired in a probe with, for example, BLACK HOLE Quencher™ 1 dye, and PULSAR 650 dye can be paired in a probe with, for example, BLACK HOLE

Quencher™ 2 dye. For example, fluorescent dyes include, but are not limited to, DAPI, 5-FAM, 6-FAM, 5(6)-FAM, 5-ROX, 6-ROX, 5,6-ROX, 5-TAMRA, 6-TAMRA, 5(6)-TAMRA SYBR, TET, JOE, VIC, HEX, R6G, Cy3, NED, Cy3.5, Texas Red, Cy5, and Cy5.5.

The methods provided herein are suitable for use with a digital analysis technique. The digital analysis can be digital polymerase chain reaction (digital PCR, DigitalPCR, dPCR, or dePCR). The dPCR can be droplet dPCR (ddPCR).

In some cases, the methods comprise using droplet dPCR (ddPCR) where an extreme high level of enhancement in sensitivity is achieved by leveraging the removal of background template through partitioning with the inherent sensitivity provided by the hot-start primer amplification system provided herein. For example, in bulk PCR reactions, the sensitivity is about 1/100 to 1/10,000, inclusive, or e.g., 1/100 to 1/1,000, as defined by mutant/(mutant+wild-type). Using ddPCR, this sensitivity is manifest in each partition, such as across 20,000 droplets, the sensitivity is about 1/1,000 to 1/100,000, inclusive.

In general, dPCR can involve spatially isolating (or partitioning) individual polynucleotides from a sample and carrying out a polymerase chain reaction on each partition. The partition can be, e.g., a well (e.g., wells of a microwell plate), capillary, dispersed phase of an emulsion, a chamber (e.g, a chamber in an array of miniaturized chambers), a droplet, or a nucleic acid binding surface. The sample can be distributed so that each partition has 0 or 1 polynucleotides. After PCR amplification, the number of partitions with or without a PCR product can be enumerated. The total number of partitions can be about, at least, or more than 500, 1000, 2000, 3000, 4000, 5000, 6000, 7000, 8000, 9000, 10,000, 11,000, 12,000, 13,000, 14,000, 15,000, 16,000, 17,000, 18,000, 19,000, 20,000, 30,000, 40,000, 50,000, 60,000, 70,000, 80,000, 90,000, 100,000, 150,000, 200,000, 500,000, 750,000, 1,000,000, 2,500,000, 5,000,000, 7,500,000, 10,000,000, 25,000,000, 50,000,000, 75,000,000, or 100,000,000. In some cases, the total number of partitions is about 1000 to about 10,000, about 10,000 to about 100,000, about 100,000 to about 1,000,000, about 1,000,000 to about 10,000,000, or about 10,000,000 to about 100,000,000. Positive and negative droplets can be counted.

In some cases, less than 0.00001, 0.00005, 0.00010, 0.00050, 0.001, 0.005, 0.01, 0.05, 0.1, 0.5, 1, 2, 2.5, 3, 3.5, 4, 4.5, 5, 6, 7, 8, 9, or 10 copies of target polynucleotide can be detected. In some cases, less than about 1, 2, 3, 4, 5, 6, 7, 8, 9, 10, 11, 12, 13, 14, 15, 20, 25, 30, 35, 40, 45, 50, 55, 60, 65, 70, 75, 80, 85, 90, 95, 100, 150, 200, 250, 300, 350, 400, 450, or 500 copies of a target polynucleotide can be detected. In some cases, the droplets described herein can be generated at a rate of greater than 1, 2, 3, 4, 5, 10, 50, 100, 200, 300, 400, 500, 600, 700, 800, 900, 1000, 2500, 5000, 10,000, 25,000, 50,000, 75,000, 100,000, 250,000, 500,000, or 1,000,000 droplets/second. In some cases, the droplets described herein can be generated at a rate of about 1 to about 10, about 10 to about 100, about 100 to about 1000, about 1000 to about 10,000, about 10,000 to about 100,000, or about 100,000 to about 1,000,000 droplets/second.

An integrated, rapid, flow-through thermal cycler device can be used in the methods according to the disclosure. See, e.g., International Application No. PCT/US2009/005317, filed Sep. 23, 2009. In such an integrated device, a capillary is wound around a cylinder that maintains 2, 3, or 4 temperature zones. As droplets flow through the capillary, they are subjected to different temperature zones to achieve thermal cycling. The small volume of each droplet results in an extremely fast temperature transition as the droplet enters each temperature zone.

A digital PCR device for use with the methods, compositions, and kits described herein can detect multiple signals (see e.g. PCT publication no WO2012109500A2, incorporated by reference herein in its entirety).

In some methods according to the disclosure, detection of DNA via amplification is by so-called “real time amplification” methods also known as quantitative PCR (qPCR) or Tagman. The basis for this method of monitoring the formation of amplification product formed during a PCR reaction with a template using oligonucleotide probes/oligos specific for a region of the template to be detected. In some embodiments, qPCR or Tagman are used immediately following a reverse-transcriptase reaction performed on isolated cellular mRNA; this variety serves to quantitate the levels of individual mRNAs during qPCR.

Taqman uses a dual-labeled fluorogenic oligonucleotide probe. The dual labeled fluorogenic probe used in such assays is typically a short (ca. 20-25 bases) polynucleotide that is labeled with two different fluorescent dyes. The 5′ terminus of the probe is typically attached to a reporter dye and the 3′ terminus is attached to a quenching dye. Regardless of labelling or not, the qPCR probe is designed to have at least substantial sequence complementarity with a site on the target mRNA or nucleic acid derived from. Upstream and downstream PCR primers that bind to flanking regions of the locus are also added to the reaction mixture. When the probe is intact, energy transfer between the two fluorophores occurs and the quencher quenches emission from the reporter. During the extension phase of PCR, the probe is cleaved by the 5′ nuclease activity of a nucleic acid polymerase such as Taq polymerase, thereby releasing the reporter from the polynucleotide-quencher and resulting in an increase of reporter emission intensity which can be measured by an appropriate detector. The recorded values can then be used to calculate the increase in normalized reporter emission intensity on a continuous basis and ultimately quantify the amount of the mRNA being amplified. mRNA levels can also be measured without amplification by hybridization to a probe, for example, using a branched nucleic acid probe, such as a QuantiGene® Reagent System from Panomics. This format of test is particularly useful for the multiplex detection of multiple genes from a single sample reaction, as each fluorophore/quencher pair attached to an individual probe may be spectrally orthogonal to the other probes used in the reaction such that multiple probes (each directed against a different gene product) can be detected during the amplification/detection reaction.

qPCR can also be performed without a dual-labeled fluorogenic probe by using a fluorescent dye (e.g. SYBR Green) specific for dsDNA that reflects the accumulation of dsDNA amplified specific upstream and downstream oligonucleotide primers. The increase in fluorescence during the amplification reaction is followed on a continuous basis and can be used to quantify the amount of mRNA being amplified.

In some embodiments, for qPCR or Tagman detection, a “pre-amplification” step is performed on cDNA transcribed from cellular RNA prior to the quantitatively monitored PCR reaction. This serves to increase signal in conditions where the natural level of the RNA/cDNA to be detected is very low. Suitable methods for pre-amplification include but are not limited LM-PCR, PCR with random oligonucleotide primers (e.g. random hexamer PCR), PCR with poly-A specific primers, and any combination thereof.

In some embodiments, for qPCR or Tagman detection, an RT-PCR step is first performed to generate cDNA from cellular RNA. Such amplification by RT-PCR can either be general (e.g. amplification with partially/fully degenerate oligonucleotide primers) or targeted (e.g. amplification with oligonucleotide primers directed against specific genes which are to be analyzed at a later step).

In some cases, any of the methods described herein can further comprise performing a sequencing assay on extension, amplification, or processing products produced according to any of the methods described herein. The sequencing assay can comprise (i) exome sequencing, (ii) sequencing a panel of genes, (iii) whole genome sequencing, (iv) sequencing by synthesis using reversible terminator chemistry, (v) pyrosequencing, (vi) nanopore sequencing, (vii) real-time single molecule sequencing, (viii) sanger sequencing, or any combination thereof. Sequencing can be performed by various systems currently available, such as, without limitation, a sequencing system by Illumina®, Pacific Biosciences (PacBio®), Oxford Nanopore®, or Life Technologies (Ion Torrent®) or other “next generation sequencing” technologies.

### EXAMPLES

### Example 1.—Optimization of Primer Concentration for ACTN3 and NRAS Mutant Stem-Loop Primer qPCR Assays

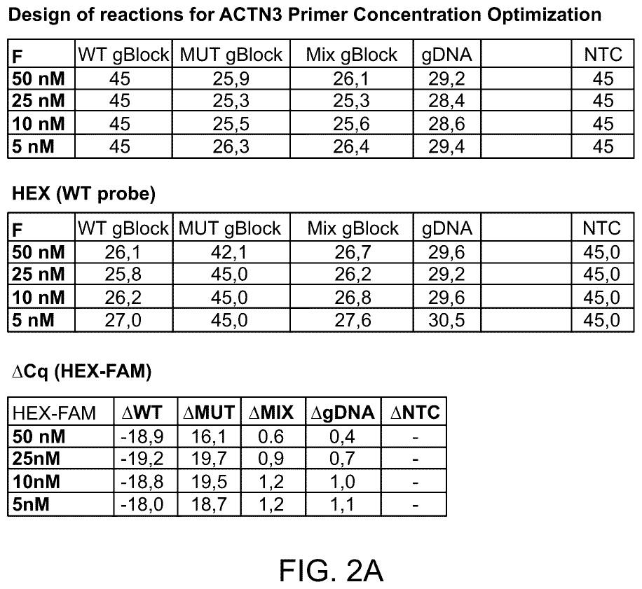

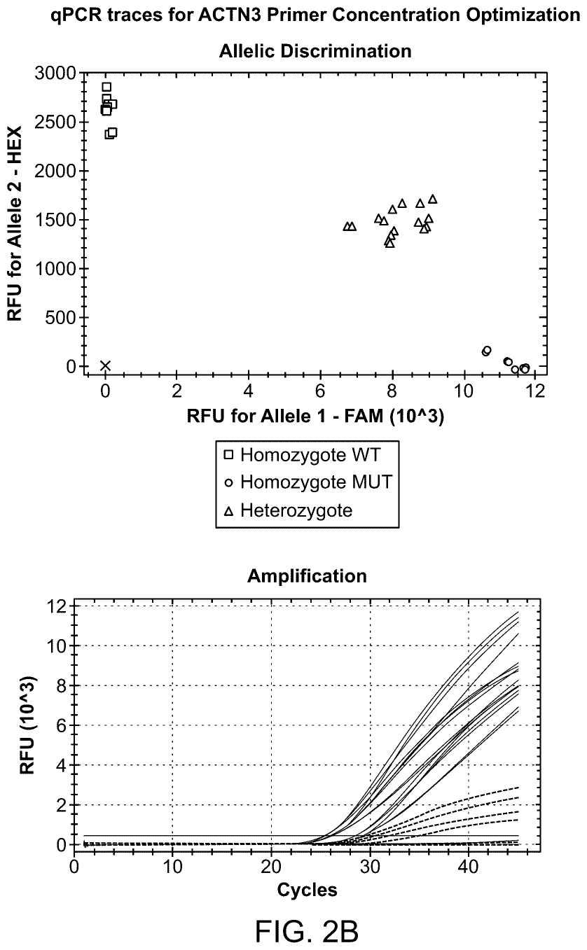

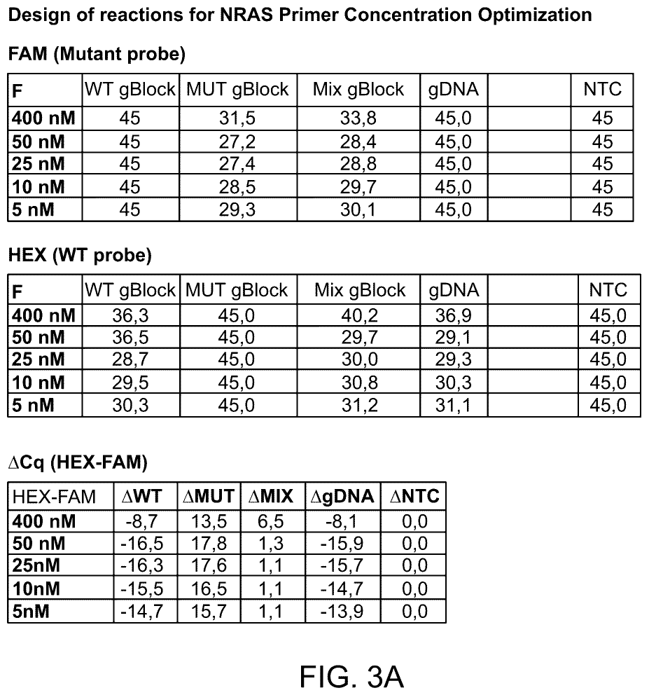

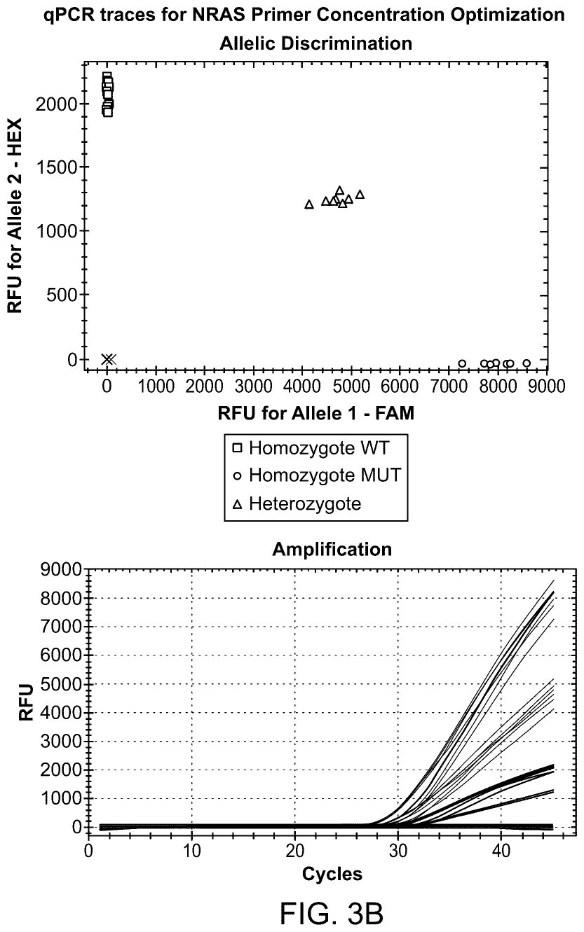

To find optimal concentration stem-loop primers, different concentrations varying from 10 nM to 200 nM of primers were evaluated. The various concentrations of primers were evaluated on 100% wild-type, 100% mutant, 50% wild-type/50% mutant, genomic DNA and negative control. The designs for ACTN3 and NRAS in Table 1 were assessed separately. Reaction contained 400 nM forward and reverse primers, 5-25 nM mutant and wild-type stem-loop primers and 200 nM of mutant and wild-type probes. qPCR was run at 95° C. for 60 seconds, followed by 45 cycles of 5 seconds at 95° C. and 30 seconds at 95° C. For better visualization of the data all negative samples were assigned an arbitrary Cq value of 45. Data were evaluated by the measured difference between Cq values for wild-type and mutant assays (ΔCq) and scatter plots of end point fluorescence values. Results of this experiment are shown in FIG. 2 and FIG. 3.

### Example 2.—Assessment of Selectivity of ACTN3 and NRAS Mutant Stem-Loop Primer qPCR Assays

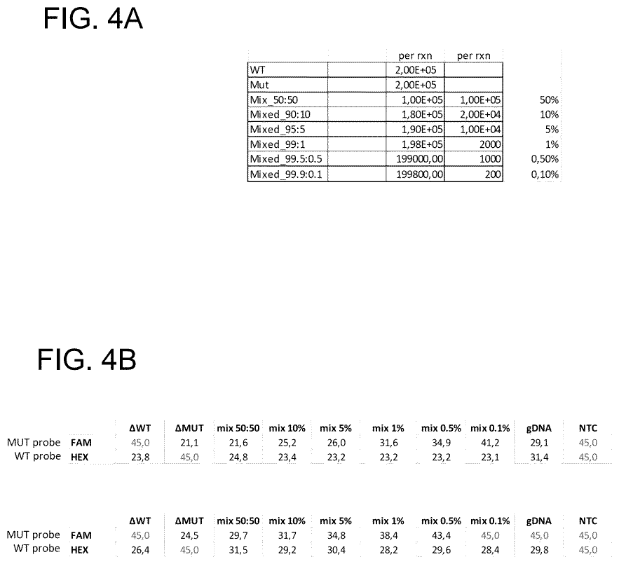

To evaluate sensitivity and specificity of the designs, different ratios of mutant gBlock DNA versus wild-type DNA was evaluated at a constant total amount of gBlock DNA, Also included for comparison was 100% wild-type, 100% mutant genomic DNA and negative control. The designs for ACTN3 and NRAS in Table 1 were assessed separately. Reaction contained 400 nM forward and reverse primers, 25 nM mutant and wild-type stem-loop primers and 200 nM of mutant and wild-type probes. qPCR was run at 95° C. for 60 seconds, followed by 45 cycles of 5 seconds at 95° C. and 30 seconds at 95° C. For better visualization of the data all negative samples were assigned an arbitrary Cq value of 45. Results of this experiment are shown in FIG. 4.

### Example 3.—KRAS Rare Sequence Variant Detection for KRAS G12R Mutation

### Reagents/Procedure

Synthetic templates for wild-type (WT) KRAS and mutant KRAS (KRAS G12R) were constructed (gBlocks™, custom ordered from IDT), one containing the WT sequence and one containing the Mutant (KRAS G12R) sequence for the assay target. The two templates were mixed at different ratios to simulate different mutation frequencies. The simulated mutation frequencies were: 50%, 5%, 1%, 0.5%, 0.1% and 0.05% mutant.

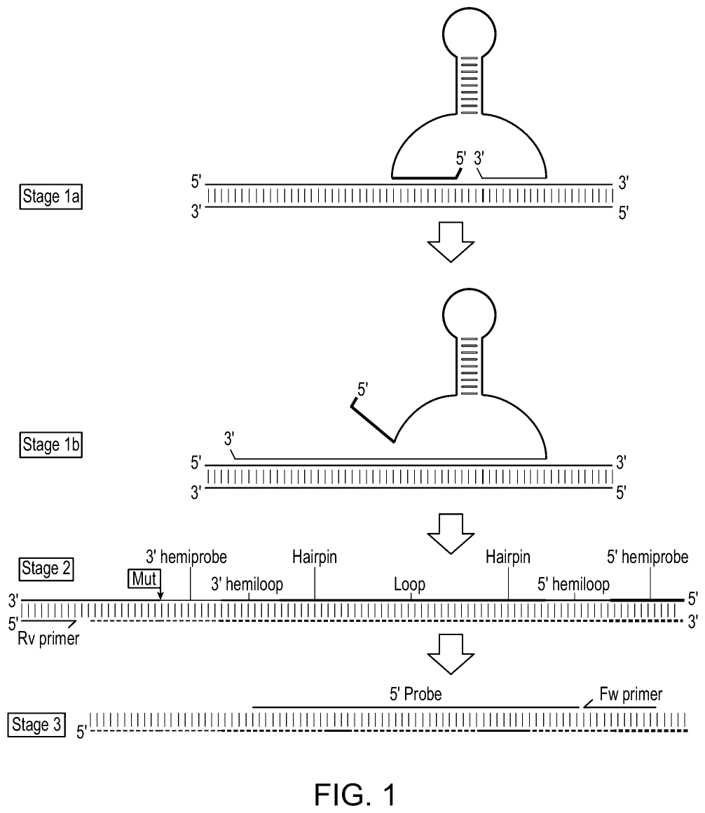

The proportion of WT and mutant DNA in each mixture was assessed by dPCR using primer designs and amplification schemes as described herein according to FIG. 1 (see Table 2). Each dPCR reaction was 40 μl and contained 10 μl QIAcuity Probe Mastermix, 800 nM forward primer, 800 nM reverse primer, 400 nM WT probe (HEX), 400 nM Mut probe (FAM), 50 nM WT TwoTail primer, 50 nM Mutant TwoTail primer, and 1600 copies/μl of synthetic template (total concentration of mutant and WT template in a sample, concentrations of the individual templates vary per samples based on the simulated mutation frequency)

TABLE 2
Primer designs used in Example 3

| Name | Sequence (DNA) |
|---|---|
| 2T WT | CTCCAACTACCACAAGTTTATTTA |
| primer | CGAAATGTTGGTACAGTGAGTACC |
| | AATATGAGGACCATCTACGCCACC |
| 2T Mut | CTCCAACTACCACAAGTTTATTTA |
| primer | CGAAATGCAGGTACAGTTGGTACC |
| | TGTCTCCACCTACGCCACG |
| WT probe | TTGGTACAGTGAGTACCAATATGA |
| | GGACCATC |
| Mut probe | CAGGTACAGTTGGTACCTGTCTCC |
| | ACC |
| Forward | CCTGCTGAAAATGACTGAA |
| primer | |
| Reverse | CCAACTACCACAAGTTTATTTAC |
| primer | |

The dPCR reactions were loaded into a QIAcuity Nanoplate 26K and cycled on the QIAcuity Digital PCR System with the following conditions: 3 minutes at 95° C., 45 cycles each comprising a 15 second denaturation at 95° C. followed by a 55° C. extension for 30 seconds. Image acquisition was performed in the FAM and HEX channel. The data was analyzed with the QIAcuity Software Suite using manual thresholding.

### Results

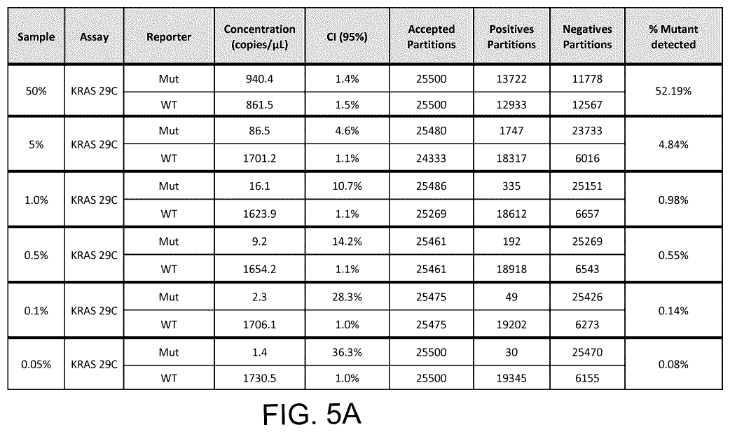

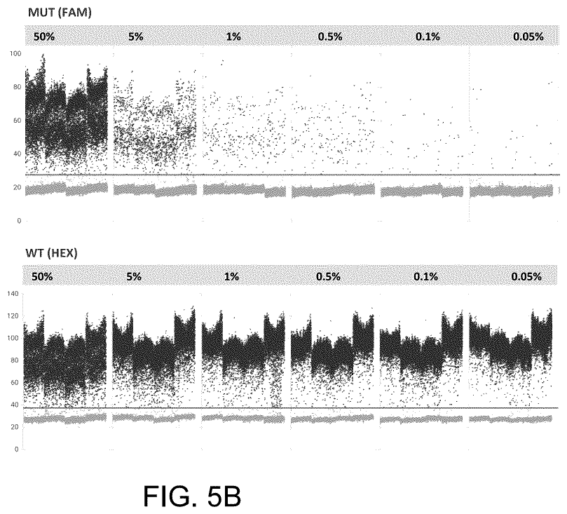

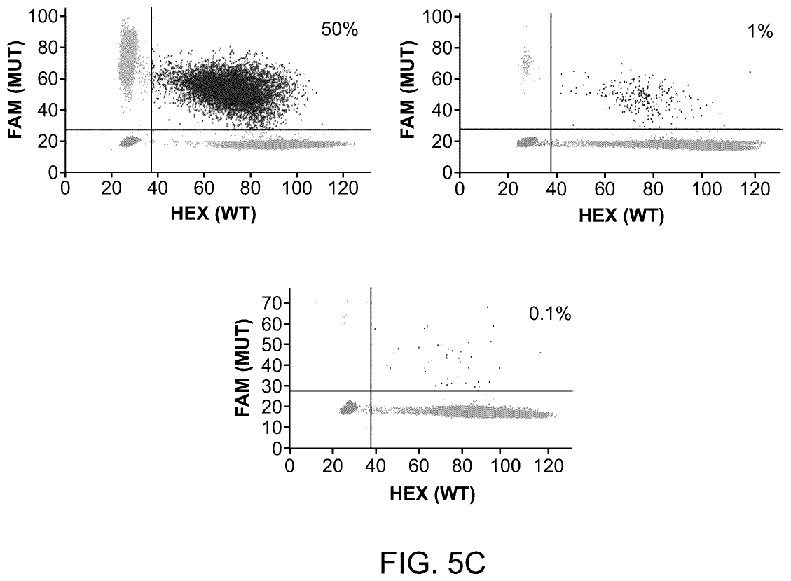

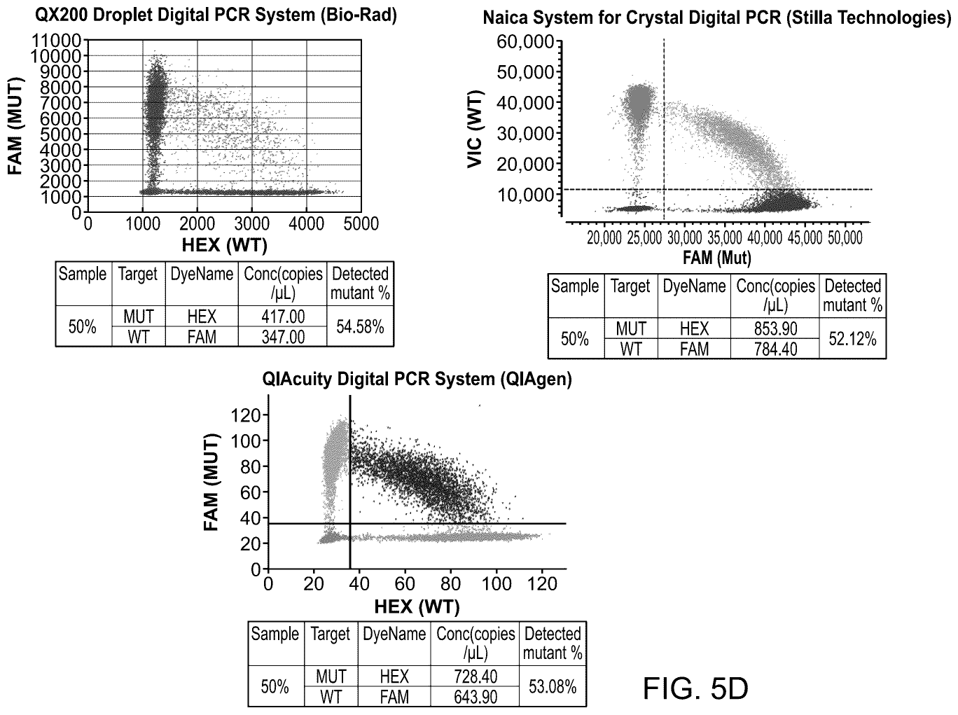

FIG. 5 depicts examples of digital PCR data of SNP detecting stem-loop primer assays from two different experiments. Panels (A-C) and Table 3 below depict results for an experiment designed to assess the sensitivity of G12R KRAS mutant detecting stem-loop primer assay on samples with different WT/mutant target template ratios (between 0.050 and 50 mutant to WT ratio) on the QIAcuity Digital PCR System. (A) depicts numerical data from the experiment, which is reproduced in Table 3 below:

TABLE 3
Results of dPCR on WT and KRAS Templates Using Primer
Designs According to the Current Disclosure

| | | | Concentration | CI | Accepted | Positives | Negatives | % Mutant |
|---|---|---|---|---|---|---|---|---|
| Sample | Assay | Reporter | (copies/μL) | (95%) | Partitions | Partitions | Partitions | detected |
| 50% | KRAS | Mut | 940.4 | 1.4% | 25500 | 13722 | 11778 | 52.19% |
| | G12R | WT | 861.5 | 1.5% | 25500 | 12933 | 12567 | |
| 5% | KRAS | Mut | 86.5 | 4.6% | 25480 | 1747 | 23733 | 4.84% |
| | G12R | WT | 1701.2 | 1.1% | 24333 | 18317 | 6016 | |
| 1.0% | KRAS | Mut | 16.1 | 10.7% | 25486 | 335 | 25151 | 0.98% |
| | G12R | WT | 1623.9 | 1.1% | 25269 | 18612 | 6657 | |
| 0.5% | KRAS | Mut | 9.2 | 14.2% | 25461 | 192 | 25269 | 0.55% |
| | G12R | WT | 1654.2 | 1.1% | 25461 | 18918 | 6543 | |
| 0.1% | KRAS | Mut | 2.3 | 28.3% | 25475 | 49 | 25426 | 0.14% |
| | G12R | WT | 1706.1 | 1.0% | 25475 | 19202 | 6273 | |
| 0.05% | KRAS | Mut | 1.4 | 36.3% | 25500 | 30 | 25470 | 0.08% |
| | G12R | WT | 1730.5 | 1.0% | 25500 | 19345 | 6155 | |

### Example 4.—KRAS Rare Sequence Variant Detection for KRAS 29T Mutation

Having assessed the ability of the 2T design according to FIG. 1 to detect a single KRAS mutation in Example 3, the performance of the method with a variety of dPCR platforms (QX200, Naica, and QIAcuity) was assessed.

### Reagents/Procedure

The amplification samples used in the experiment comprised synthetic templates (gBlocks™, custom ordered from IDT) for mutant (KRAS 29T) and WT KRAS. Two templates were used in the experiment per assay, one containing the WT sequence and one containing the Mutant sequence for the assay target.

For the QX200 platform: Each dPCR reaction was 20 μl and contained 10 μl ddPCR Supermix for Probes (No dUTP), 800 nM forward primer, 800 nM reverse primer, 400 nM WT probe (HEX), 400 nM Mut probe (FAM), 25 nM WT TwoTail primer, 25 nM Mutant TwoTail primer, 500 copies/μl of synthetic WT template and 500 copies/μl of synthetic Mutant template. The dPCR reaction mix was loaded into the Bio-Rad QX100 Droplet Generator along with Droplet Generation Oil for Probes, and droplets were formed following the manufacturer's instructions. The droplets were transferred to a 96-well reaction plate and heat-sealed with pierceable foil. The sealed plate was cycled in a thermocycler according to the following conditions: 4 minutes at 95° C., 45 cycles each comprising a 30 second denaturation at 94° C. followed by a 53° C. extension for 60 seconds, and final step of 10 min at 95° C. The plate was incubated at 4° C. for 1 h prior to detection with the QX200 Droplet Reader in the FAM and HEX acquisition channels. The data was analyzed with the QuantaSoft™ Analysis Pro Software using manual thresholding.

For the Naica platform: Each dPCR reaction was 25 μl and contained 12.5 μl TATAA Probe Grandmaster Mix, 600 nM forward primer, 600 nM reverse primer, 400 nM WT probe (HEX), 400 nM Mut probe (FAM), 25 nM WT TwoTail primer, 25 nM Mutant TwoTail primer, 100 nM fluorescein, 1000 copies/μl of synthetic WT template and 1000 copies/μl of synthetic Mutant template. The dPCR reaction mix was loaded into a Sapphire chip and cycled on the cycler unit of the Naica System according to the following conditions: 4 minutes at 95° C., 45 cycles each comprising a 10 second denaturation at 95° C. followed by a 55° C. extension for 30 seconds. The Sapphire chip was transferred to the reading unit of the Naica System and detected in the FAM and HEX channel. The data was analyzed with the Naica Crystal Reader and Nacia Crystal Miner Software using manual thresholding.

QIAcuity platform: Each dPCR reaction was 40 μl and contained 10 μl QIAcuity Probe Mastermix, 800 nM forward primer, 800 nM reverse primer, 400 nM WT probe (HEX), 400 nM Mut probe (FAM), 25 nM WT TwoTail primer, 25 nM Mutant TwoTail primer and 800 copies/μl of synthetic WT template and 800 copies/μl of synthetic Mutant template. The dPCR reaction mix was loaded into a QIAcuity Nanoplate 26K and cycled on the QIAcuity Digital PCR System with the following conditions: 3 minutes at 95° C., 45 cycles each comprising a 15 second denaturation at 95° C. followed by a 56° C. extension for 30 seconds. Image acquisition was performed in the FAM and HEX channel. The data was analyzed using the QIAcuity Software Suite manual thresholding.

TABLE 4
Primer designs used in Example 4

| Name | Sequence (DNA) |
|---|---|
| 2T WT | CTCCAACTACCACAAGTTTATTTA |
| primer | CGAAATGTTGGTACAGTGAGTACC |
| | AATATGAGGACCATCTACGCCACC |
| 2T Mut | CTCCAACTACCACAAGTTTATTTA |
| primer | CGAAATGCAGGTACAGTTGGTACC |
| (KRAS 29T) | TGTCTCCACCTACGCCACA |
| WT probe | TTGGTACAGTGAGTACCAATATGA |
| | GGACCATC |
| Mut probe | CAGGTACAGTTGGTACCTGTCTCC |
| (KRAS 29T) | ACC |
| Forward | CCTGCTGAAAATGACTGAA |
| primer | |
| Reverse | CCAACTACCACAAGTTTATTTAC |
| primer | |

### Results

FIG. 5 panel (D) depicts results from a set of experiments designed to assess the function of a G12R KRAS mutant detecting stem-loop primer assay on different dPCR platforms. The results depicted are 2D amplitude plots and abbreviated numerical result tables from the same assay run on samples with 50% WT and 50% mutant target template on three different dPCR platforms: QX200 Droplet Digital PCR System, Naica System for Crystal Digital PCR and QIAcuity Digital PCR System. As can be seen by comparison of the plots and associated data, the 2T design scheme had similar performance among all three platforms.

### Example 5.—Assessment of Performance of Generic Stem-Loop Sequences for 2T Primers

Having assessed the usefulness of the 2T design in one instance of WT/mutant DNA detection, the general performance of two different stem loop sequences with a variety of different 5′ and 3′ hemiprobes was assessed (See FIG. 4 panel A). For wild-type detection, the generic 2T primer sequence used was 5′-hemiprobe-TTACGAAATGTTGGTACAGTGAGTACCAATATGAGGACCATC-3′hemiprobe (SEQ ID NO: 21), while the generic 2T primer sequence used for mutant detection was 5′-hemiprobe-TTACGAAATGCAGGTACAGTTGGTACCTGTCTCCACC-3′hemiprobe (SEQ ID NO: 22). Assessments were performed for detection of KRAS G12R and NRAS Q61R.

### Reagents/Procedure

For detection of KRAS G12R, procedures were performed as in Example 3 and primers were as described in Example 3.

For detection of NRAS Q61R, NRAS samples for amplification comprised synthetic templates (gBlocks™, custom ordered from IDT). Two templates were used in the experiment, one containing the WT sequence and one containing the Mutant sequence for the assay target. The two templates were mixed at different ratios to simulate different mutation frequencies. The simulated mutation frequencies were: 50%, 10%, 5%, 2.5%, 1.0%, 0.5% and 0.1% mutant.

Each dPCR reaction was 40 μl and contained 10 μl QIAcuity Probe Mastermix, 400 nM forward primer, 400 nM reverse primer, 200 nM WT probe (HEX), 200 nM Mut probe (FAM), 25 nM WT TwoTail primer, 24 nM Mutant TwoTail primer and 2400 copies/μl of synthetic template (total concentration of mutant and WT template in a sample, concentrations of the individual templates vary per sample depending on the simulated mutation frequency).

The dPCR reaction mix was loaded into a QIAcuity Nanoplate 26K and cycled on the QIAcuity Digital PCR System with the following conditions: 3 minutes at 95° C., 45 cycles each comprising a 15 second denaturation at 95° C. followed by a 60° C. extension for 30 seconds. Image acquisition was performed in the FAM and HEX channel. The data was analyzed using the QIAcuity Software Suite using manual thresholding.

TABLE 5
Primer Designs used in Example 5
for NRAS Q61R detection

| Name | Sequence (DNA) |
|---|---|
| 2T WT | GAGTACAGTGCCATGAGAGACTT |
| primer | ACGAAATGTTGGTACAGTGAGTA |
| | CCAATATGAGGACCATCAGCTGG |
| | ACA |
| 2T Mut | GAGTACAGTGCCATGAGAGACTT |
| primer | ACGAAATGCAGGTACAGTTGGTA |
| | CCTGTCTCCACCAGCTGGACG |
| WT probe | TTGGTACAGTGAGTACCAATATG |
| | AGGACCATC |
| Mut probe | CAGGTACAGTTGGTACCTGTCTC |
| | CACC |
| Forward | GCAAATACACAGAGGAAGCC |
| primer | |
| Reverse | TGCCATGAGAGACTTACGAAAT |
| primer | |

### Results

For detection of KRAS WT and mutant, results are shown in Table 6 below

TABLE 6
Detection of KRAS WT/G12R mutant with generic stem loop sequences

| | | | Concentration | CI | Accepted | Positives | Negatives | % Mutant |
|---|---|---|---|---|---|---|---|---|
| Sample | Assay | Reporter | (copies/μL) | (95%) | Partitions | Partitions | Partitions | detected |
| 50% | KRAS | Mut | 940.4 | 1.4% | 25500 | 13722 | 11778 | 52.19% |
| | G12R | WT | 861.5 | 1.5% | 25500 | 12933 | 12567 | |
| 5% | KRAS | Mut | 86.5 | 4.6% | 25480 | 1747 | 23733 | 4.84% |
| | G12R | WT | 1701.2 | 1.1% | 24333 | 18317 | 6016 | |
| 1.0% | KRAS | Mut | 16.1 | 10.7% | 25486 | 335 | 25151 | 0.98% |
| | G12R | WT | 1623.9 | 1.1% | 25269 | 18612 | 6657 | |
| 0.5% | KRAS | Mut | 9.2 | 14.2% | 25461 | 192 | 25269 | 0.55% |
| | G12R | WT | 1654.2 | 1.1% | 25461 | 18918 | 6543 | |
| 0.1% | KRAS | Mut | 2.3 | 28.3% | 25475 | 49 | 25426 | 0.14% |
| | G12R | WT | 1706.1 | 1.0% | 25475 | 19202 | 6273 | |
| 0.05% | KRAS | Mut | 1.4 | 36.3% | 25500 | 30 | 25470 | 0.08% |
| | G12R | WT | 1730.5 | 1.0% | 25500 | 19345 | 6155 | |

As can be seen with the results in Table 6, the % mutant detected aligned closely (e.g. within 100%) with the amount placed in the sample (compare “sample” and “% mutant detected” columns).

TABLE 7
Detection of NRAS WT/mutant with generic stem loop sequences

| | | | Concentration | CI | Accepted | Positives | Partitions | % Mutant |
|---|---|---|---|---|---|---|---|---|
| Sample | Assay | Reporter | (copies/μL) | (95%) | Partitions | Partitions | Negatives | detected |
| 50% | NRAS | Mut | 1318 | 1.30% | 25477 | 15531 | 9946 | 52.74% |
| | | WT | 1181.1 | 1.30% | 25477 | 14510 | 10967 | |
| 10% | NRAS | Mut | 241.3 | 3.00% | 25469 | 3985 | 21484 | 9.18% |
| | | WT | 2386.5 | 0.90% | 25469 | 20736 | 4733 | |
| 5% | NRAS | Mut | 122.8 | 4.20% | 25476 | 2131 | 23345 | 4.41% |
| | | WT | 2664.3 | 0.90% | 25476 | 21645 | 3831 | |
| 2.5% | NRAS | Mut | 64.23 | 5.70% | 25480 | 1140 | 24340 | 2.44% |
| | | WT | 2573 | 0.90% | 25480 | 21407 | 4073 | |
| 1.0% | NRAS | Mut | 28.19 | 8.60% | 25472 | 512 | 24960 | 1.08% |
| | | WT | 2588.7 | 0.90% | 25460 | 21514 | 3946 | |
| 0.5% | NRAS | Mut | 22.18 | 9.60% | 25449 | 415 | 25034 | 0.78% |
| | | WT | 2813.2 | 0.90% | 25436 | 22276 | 3160 | |
| 0.1% | NRAS | Mut | 8.232 | 16.00% | 25493 | 150 | 25343 | 0.30% |
| | | WT | 2774.5 | 0.90% | 25493 | 22005 | 3488 | |

As can be seen with the results in Table 7, the % mutant detected aligned closely (e.g. within 100%) with the amount placed in the sample (compare “sample” and “% mutant detected” columns).

### Example 6.—Detection of Multiple Different KRAS Mutants in Single-Plex Assays

Having demonstrated the 2T design of FIG. 1 was highly effective for detection of a single mutant of KRAS, probe combinations to detect multiple different KRAS mutants were designed and tested.

### Reagents/Procedure

The samples used in the experiment for amplification bearing WT, 29T, 29C, 29A, 30T, 30C comprised synthetic templates (gBlocks™, custom ordered from TDT). Two templates were used in the experiment per assay, one containing the WT sequence and one containing the Mutant sequence for the assay target.

Each dPCR reaction was 12 μl and contained 3 μl QIAcuity Probe Mastermix, 800 nM forward primer, 800 nM reverse primer, 400 nM WT probe (HEX), 400 nM Mut probe (FAM), 100 nM WT TwoTail primer, 50 nM Mutant TwoTail primer, 500 copies/μl of synthetic WT template and 500 copies/μl of synthetic Mutant template.

The dPCR reaction mix was loaded into a QIAcuity Nanoplate 8.5K and cycled on the QIAcuity Digital PCR System with the following conditions: 3 minutes at 95° C., 45 cycles each comprising a 15 second denaturation at 95° C. followed by a 55° C. extension for 30 seconds. Image acquisition was performed in the FAM and HEX channel. The data was analyzed using the QIAcuity Software Suite using manual thresholding.

TABLE 8
Primer Designs used in Example 6 for
KRAS 29T detection

| Name | Sequence (DNA) |
|---|---|
| 2T WT primer | CTCCAACTACCACAAGTTTATTTA |
| KRAS 29T | CGAAATGTTGGTACAGTGAGTACC |
| | AATATGAGGACCATCTACGCCACC |
| 2T Mut primer | CTCCAACTACCACAAGTTTATTTA |
| KRAS 29T | CGAAATGCAGGTACAGTTGGTACC |
| | TGTCTCCACCTACGCCACA |
| WT probe | TTGGTACAGTGAGTACCAATATGA |
| KRAS 29T | GGACCATC |
| Mut probe | CAGGTACAGTTGGTACCTGTCTCC |
| KRAS 29T | ACC |
| Forward | CCTGCTGAAAATGACTGAA |
| primer KRAS | |
| 29T | |
| Reverse | CCAACTACCACAAGTTTATTTAC |
| primer KRAS | |
| 29T | |

TABLE 9
Primer Designs used in Example 6
for KRAS 29C detection

| Name | Sequence (DNA) |
|---|---|
| 2T WT primer | CTCCAACTACCACAAGTTTATTTA |
| KRAS 29C | CGAAATGTTGGTACAGTGAGTACC |
| | AATATGAGGACCATCTACGCCACC |
| 2T Mut primer | CTCCAACTACCACAAGTTTATTTA |
| KRAS 29C | CGAAATGCAGGTACAGTTGGTACC |
| | TGTCTCCACCTACGCCACG |
| WT probe | TTGGTACAGTGAGTACCAATATGA |
| KRAS 29C | GGACCATC |
| Mut probe | CAGGTACAGTTGGTACCTGTCTCC |
| KRAS 29C | ACC |
| Forward | CCTGCTGAAAATGACTGAA |
| primer KRAS | |
| 29C | |
| Reverse | CCAACTACCACAAGTTTATTTAC |
| primer KRAS | |
| 29C | |

TABLE 10
Primer Designs used in Example 6
for KRAS 29A detection

| Name | Sequence (DNA) |
|---|---|
| 2T WT primer | CTCCAACTACCACAAGTTTATTTA |
| KRAS 29A | CGAAATGTTGGTACAGTGAGTACC |
| | AATATGAGGACCATCTACGCCACC |
| 2T Mut primer | CTCCAACTACCACAAGTTTATTTA |
| KRAS 29A | CGAAATGCAGGTACAGTTGGTACC |
| | TGTCTCCACCTACGCCACT |
| WT probe | TTGGTACAGTGAGTACCAATATGA |
| KRAS 29A | GGACCATC |
| Mut probe | CAGGTACAGTTGGTACCTGTCTCC |
| KRAS 29A | ACC |
| Forward | CCTGCTGAAAATGACTGAA |
| primer KRAS | |
| 29A | |
| Reverse | CCAACTACCACAAGTTTATTTAC |
| primer KRAS | |
| 29A | |

TABLE 11
Primer Designs used in Example 6
for KRAS 30T detection

| Name | Sequence (DNA) |
|---|---|
| 2T WT primer | CTCCAACTACCACAAGTTTATTTA |
| KRAS 30T | CGAAATGTTGGTACAGTGAGTACC |
| | AATATGAGGACCATCTACGCCACC |
| 2T Mut | CTCCAACTACCACAAGTTTATTTA |
| primer | CGAAATGCAGGTACAGTTGGTACC |
| KRAS 30T | TGTCTCCACCTACGCCAA |
| WT probe | TTGGTACAGTGAGTACCAATATGA |
| KRAS 30T | GGACCATC |
| Mut probe | CAGGTACAGTTGGTACCTGTCTCC |
| KRAS 30T | ACC |
| Forward | CCTGCTGAAAATGACTGAA |
| primer KRAS | |
| 30T | |
| Reverse | CCAACTACCACAAGTTTATTTAC |
| primer KRAS | |
| 30T | |

TABLE 12
Primer Designs used in Example 6
for KRAS 30C detection

| Name | Sequence (DNA) |
|---|---|
| 2T WT primer | CTCCAACTACCACAAGTTTATTTA |
| (KRAS 30C) | CGAAATGTTGGTACAGTGAGTACC |
| | AATATGAGGACCATCTACGCCACC |
| 2T Mut primer | CTCCAACTACCACAAGTTTATTTA |
| (KRAS 30C) | CGAAATGCAGGTACAGTTGGTACC |
| | TGTCTCCACCTACGCCAG |
| WT probe | TTGGTACAGTGAGTACCAATATGA |
| (KRAS 30C) | GGACCATC |
| Mut probe | CAGGTACAGTTGGTACCTGTCTCC |
| (KRAS 30C) | ACC |
| Forward | CCTGCTGAAAATGACTGAA |
| primer (KRAS | |
| 30C) | |
| Reverse | CCAACTACCACAAGTTTATTTAC |
| primer (KRAS | |
| 30C) | |

### Results

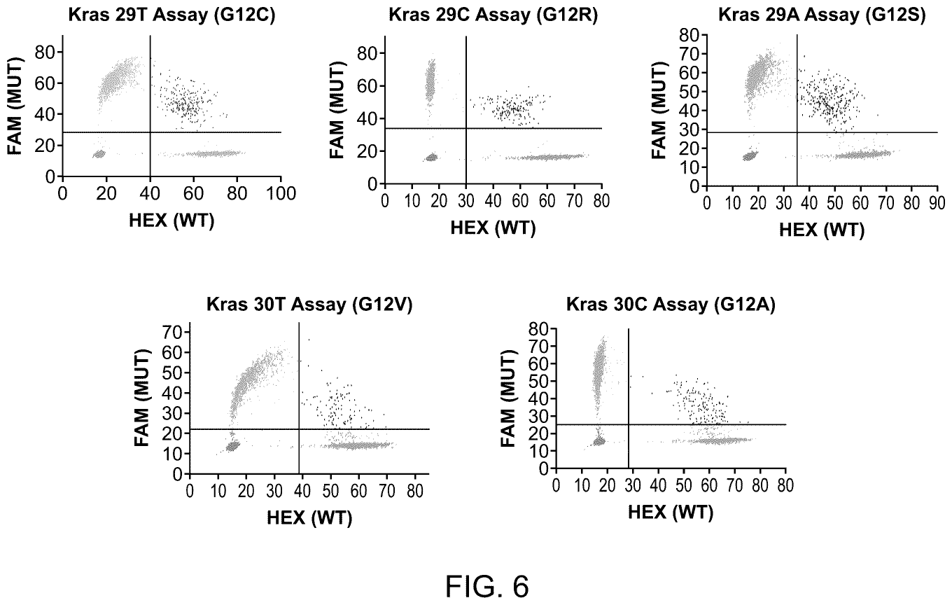

FIG. 6 depicts 2D amplification plots for the five different KRAS mutant detecting stem-loop assays all using same the generic stem-loop sequences and complementary probes. As can be seen by the segregation of point son the plots, all five mutant detection assays perform highly with minimal incorrect overlap of points.

### Example 7.—Detection of Multiple Different KRAS Mutants Using a Common Stem-Loop Sequence

Having demonstrated the 2T design of FIG. 1 was highly effective for detection of a multiple different mutations for KRAS individually, the ability to discriminate multiple mutations in with a common stem-loop sequence was assessed.

### Reagents/Procedure

The samples used in the experiment for amplification comprised synthetic templates (gBlocks™, custom ordered from TDT). Six templates were used, one containing the WT sequence and five separate fragment each containing one mutant sequence (G12C, G12R, G12S, G12V and G12A mutations).

Each dPCR reaction was 12 μl and contained 3 μl QIAcuity Probe Mastermix, 800 nM forward primer, 800 nM reverse primer, 400 nM WT probe (HEX), 400 nM Mut probe (FAM), 50 nM WT TwoTail primer, 20 nM 29T Mutant TwoTail primer, 20 nM 29C Mutant TwoTail primer, 20 nM 29A Mutant TwoTail primer, 20 nM 30T Mutant TwoTail primer, 20 nM 30C Mutant TwoTail primer, 1000 copies/μl of WT template (WT sample) or 500 copies/μl of synthetic WT template and 500 copies/μl of one of the five synthetic Mutant templates (50% samples—sample names indicate which mutant template was used for the respective samples).

The dPCR reactions were loaded into a QIAcuity Nanoplate 8.5K and cycled on the QIAcuity Digital PCR System with the following conditions: 3 minutes at 95° C., 45 cycles each comprising a 15 second denaturation at 95° C. followed by a 55° C. extension for 30 seconds. Image acquisition was performed in the FAM and HEX channel. The data was analyzed using the QIAcuity Software Suite using manual thresholding.

TABLE 13
Primer Designs used in Example 7 for
detection of multiple different KRAS
mutants using a common stem-loop
sequence

| Name | Sequence (DNA) |
|---|---|
| 2T WT | CTCCAACTACCACAAGTTTATTTACGAAATGTTGGTACAGTGAGTACCAA |
| primer | TATGAGGACCATCTACGCCACC |
| 2T 29T | CTCCAACTACCACAAGTTTATTTACGAAATGCAGGTACAGTTGGTACCTG |
| Mut | TCTCCACCTACGCCACA |
| primer | |
| 2T 29C | CTCCAACTACCACAAGTTTATTTACGAAATGCAGGTACAGTTGGTACCTG |
| Mut | TCTCCACCTACGCCACG |
| primer | |
| 2T 29A | CTCCAACTACCACAAGTTTATTTACGAAATGCAGGTACAGTTGGTACCTG |
| Mut | TCTCCACCTACGCCACT |
| primer | |
| 2T 30T | CTCCAACTACCACAAGTTTATTTACGAAATGCAGGTACAGTTGGTACCTG |
| Mut | TCTCCACCTACGCCAA |
| primer | |
| 2T 30C | CTCCAACTACCACAAGTTTATTTACGAAATGCAGGTACAGTTGGTACCTG |
| Mut | TCTCCACCTACGCCAG |
| primer | |
| WT | TTGGTACAGTGAGTACCAATATGAGGACCATC |
| probe | |
| Mut | CAGGTACAGTTGGTACCTGTCTCCACC |
| probe | |
| Forward | CCTGCTGAAAATGACTGAA |
| primer | |
| Reverse | CCAACTACCACAAGTTTATTTAC |
| primer | |

### Results

The results are shown in Table 14 below.

TABLE 14
Detection of multiple KRAS mutants with generic stem loop sequences

| | | | Concentration | CI | Accepted | Positives | Negatives | Detected |
|---|---|---|---|---|---|---|---|---|
| Sample | MMX | Reporter | (copies/μL) | (95%) | Partitions | Partitions | Partitions | Mutant % |
| WT | Screening | Mut | 0.39 | 274.40% | 8253 | 1 | 8252 | 0.04% |
| | Assay | WT | 976.4 | 3.90% | 8253 | 2161 | 6092 | |
| | KRAS | | | | | | | |
| 50% | Screening | Mut | 467.7 | 5.70% | 8257 | 1101 | 7156 | 47.37% |
| G12C | Assay | WT | 519.7 | 5.40% | 8257 | 1214 | 7043 | |
| | KRAS | | | | | | | |
| 50% | Screening | Mut | 528.1 | 5.30% | 8262 | 1245 | 7017 | 50.11% |
| G12R | Assay | WT | 525.8 | 5.30% | 8262 | 1240 | 7022 | |
| | KRAS | | | | | | | |
| 50% | Screening | Mut | 529.1 | 5.20% | 8225 | 1290 | 6935 | 48.44% |
| G12S | Assay | WT | 563.2 | 5.10% | 8225 | 1366 | 6859 | |
| | KRAS | | | | | | | |
| 50% | Screening | Mut | 258.6 | 7.70% | 8274 | 620 | 7654 | 32.48% |
| G12V | Assay | WT | 537.7 | 5.40% | 8274 | 1237 | 7037 | |
| | KRAS | | | | | | | |
| 50% | Screening | Mut | 460.6 | 5.60% | 8242 | 1126 | 7116 | 46.43% |
| G12A | Assay | WT | 531.5 | 5.20% | 8242 | 1285 | 6957 | |
| | KRAS | | | | | | | |

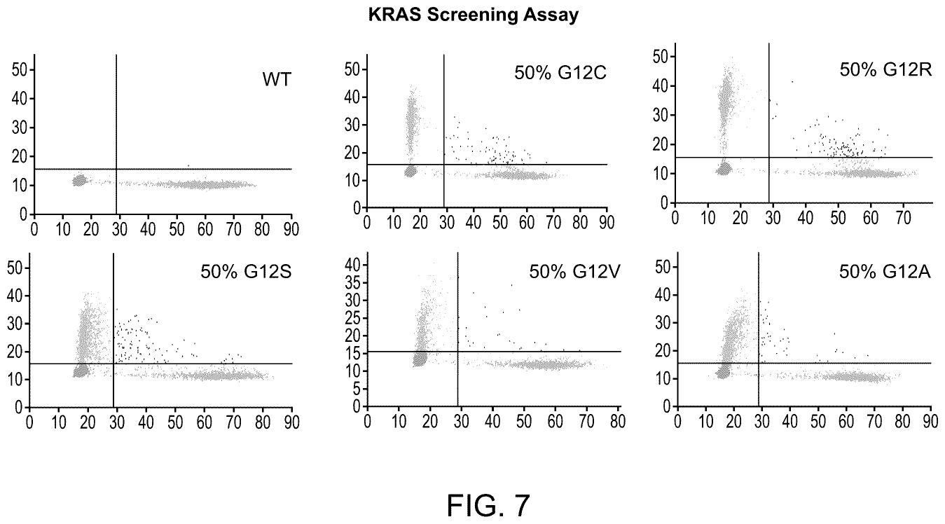

As can be seen from the summarized data, the amount of detected mutant for each of the mutations aligns closely with the actual value of 50% demonstrating that the primer sets with common stem-loop sequences are highly effective for detection of all the mutants in Table 14. FIG. 7 shows 2D amplitude plots for the same experiment, demonstrating graphically that the 2T method with common stem loop primers performs well for discrimination of multiple mutants.

### Example 8.—Distinguishing Between Methylated and Unmethylated DNA Using 2-Tailed Primers

Having observed that the 2-tailed design of FIG. 1 was effective for detecting mutant DNA, primers were designed to distinguish between unmethylated and methylated DNA based on standard protocol using bisulphite treatment, which also introduces base pair changes in DNA by converting non-methylated cytosines to uracil (e.g. causing a change from a GC base-pair to an AT base-pair in a subsequent PCR reaction). Methylated cytosines are in eukaryotic DNA found in 5′-CG-3′ dinucleotide repeats, which are particularly rich in many regions of interest known as CpG-islands.

As 2T-primers target the CG-dinucleotide steps themselves, whose methylation status is interrogated, the two-tailed hemiprobe method presents obvious advantages versus traditional PCR amplification.

CORO6 is a gene which has been documented to contain CpG islands that are hypermethylated in cardiomyocytes (see e.g. “Heart-specific DNA methylation analysis in plasma for the investigation of myocardial damage”, Ren et al., 2022, which is incorporated by reference in its entirety herein). As such, heart specific DNA can be distinguished from DNA from other tissues—for example in cfDNA from blood—by using a methylation sensitive PCR assay.

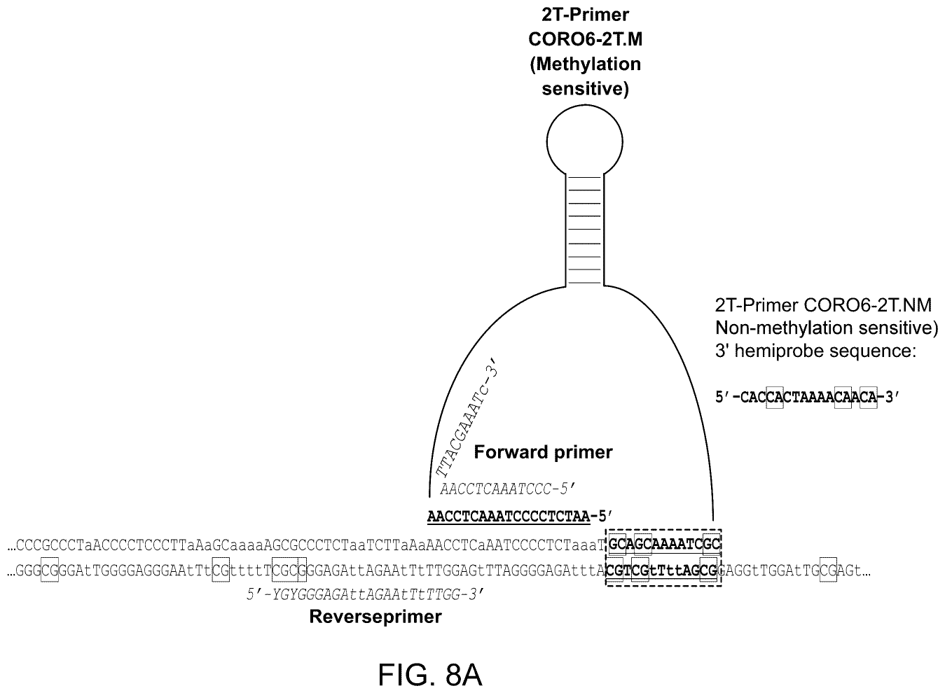

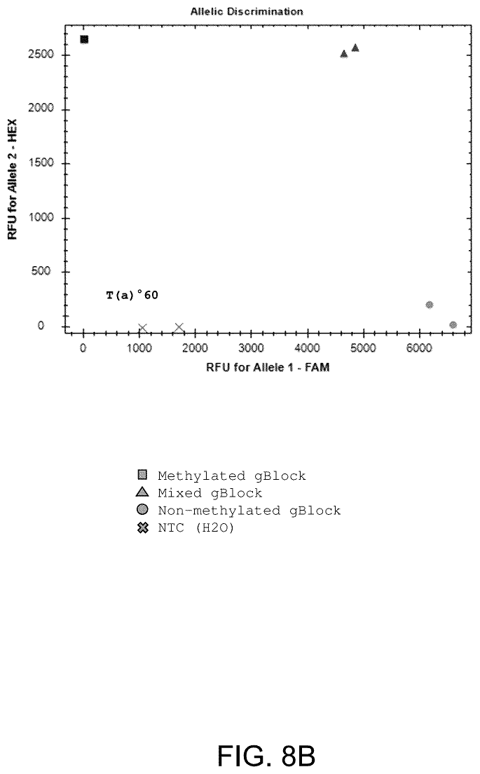

FIG. 8 panel A shows a schematic of a methylation-detecting 2T-primer (CORO6-2T.M) designed to target the CORO6 gene, with hemiprobes in black text (bold/underlined) and stem loop sequence and arms in dark grey lines, the target sequence in black text, the extended 2T-primer sequence (in grey text), the reverse and forward primer sequence (in black italics). The probe (not shown) binds selectively to the complement of the stem loop sequence and arms of the 2T-primer. The target DNA has small letters on original cytosine-sites that via bisulfite-treatment may turn into uracils (represented in the figure and in synthetic DNA sequences as thymines), while methylated CpG-sites have grey highlight (which in non-methylated DNA can be represented by TG).

The 3′ hemiprobe of the 2T-primers were designed to interrogate three CpG-sites in the CpG-island of CORO6 (the dashed box). As the 3′ hemiprobe is very short (13 bp for 2T-primer detecting methylated DNA, 16 bp for 2T primer detecting non-methylated DNA (see CORO6-2T.NM 3′-hemiprobe in figure)), the length of the interrogated DNA sequence can be kept short, which is an advantage when working with highly fragmented DNA material, such as cfDNA and bisulfite treated DNA. Furthermore, the short 3′ hemiprobe provides for discrimination between methylated and non-methylated sequence, in particular as it spans three CpG-sites.

### Reagents/Procedure

An experiment was performed to evaluate the designed CORO6-assay, the result of which is shown in FIG. 8 panel 13. The template used in the experiment comprised synthetic templates (gBlocks™, IDT), one representing the methylated CORO6 sequence (Methylated gBlock, squares in figure) and one containing the Non-methylated CORO6 sequence (Non-methylated gBlock). The template DNA was ordered in such a way that all cytosines appearing alone (not in a CpG dinucleotide) in the sequence was converted into thymine to represent uracils formed after bisulfite-treatment. Cytosines occurring in CpG-dinucleotides were left unchanged in the sequence representing methylated gBlock, while they were changed into thymines in the sequence representing non-methylated gBlock. The two templates were mixed at a 50/50 ratio (Mixed gBlock, triangles in figure) to simulate a tissue with mixed methylation pattern, such as heart tissue (see Ren et at). A no-template control was also included (NTC (1-120), crosses in figure). The template amount was 2E5 copies/reaction per target sequence.

The reaction volume was 10 μl, and contained TATAA Probe GrandMaster Mix (1×), 400 nM forward and reverse primer, 25 nM CORO6.2T-M primer (methylation detecting), 25 nM CORO6.2T-NM primer (non-methylation detecting), 200 nM HEX probe (binding to complement of 2T-M primer), 200 nM FAM probe (binding to complement of 2T-NM primer).

The qPCR reactions were cycled on a BioRad CFX384 with the following thermocycling program: 1 minute at 95° C., 45 cycles each comprising a 5 second denaturation at 95° C. followed by a 60° C. extension for 30 seconds. Image acquisition was performed in the FAM and HEX channel. The data was analyzed with the CFX Maestro Software using automatic thresholding (single threshold) and baseline adjustment (baseline subtracted curve fit).

The oligonucleotide sequences (5′4→3′) used in the experiment are listed in the table below, sequences complementary to probe binding sites are shown for 2T-primer in underlined text:

| Oligo | Sequence |
|---|---|
| CORO6.2T-M | AATCTCCCCTAAACTCCAATT |
| | ACGAAATGTACTAGCGGCAAG |
| | CTAGTGCTAGACTTGACACCG |
| | CTAAAACGACG |
| CORO6.2T-NM | AATCTCCCCTAAACTCCAATT |
| | ACGAAATGCAGGTACAGTTGG |
| | TACCTGTCTCCACCCTCCACT |
| | AAAACAACA |
| 2T-Universal HEX | TACTAGCGGCAAGCTAGTGCT |
| (methylation) | AGACTT |
| probe | |
| 2T-Universal FAM | CAGGTACAGTTGGTACCTGTC |
| (non-methylation) | TCCACC |
| probe | |
| CORO6.F | CCCTAAACTCCAATTACGAAA |
| | TG |
| CORO6.R-M | CGCGGGAGATTAGAATTTTTG |
| CORO6.R-NM | TGTGGGAGATTAGAATTTTTGG |

### Results

The graph in FIG. 8 panel B show allelic discrimination performance of the 2T-assay using the components from panel A in qPCR on synthetic gBlock sequences representing methylated DNA, non-methylated DNA, mixed methylated/non-methylated DNA and a no template control (NTC). At an annealing temperature of 60° C., a clear distinction of different type of template DNA is made while a limited false-positive signal observed in the NTC is limited to the FAM-channel (detecting non-methylated DNA), which may not interfere with detection of methylated DNA.

### Example 9.—Interrogating Methylation Status of FAM101A Using 2T-PCR

FAM101A is a gene which contain CpG-sites which have a low degree of methylation in cardiac tissue compared to other tissues (see e.g., “Non-invasive detection of human cardiomyocyte death using methylation patterns of circulating DNA”, Zemmour et al., 2018, which is incorporated by reference in its entirety herein). As such, FAM101A heart specific DNA can be distinguished in cfDNA from blood by using a methylation sensitive PCR assay.

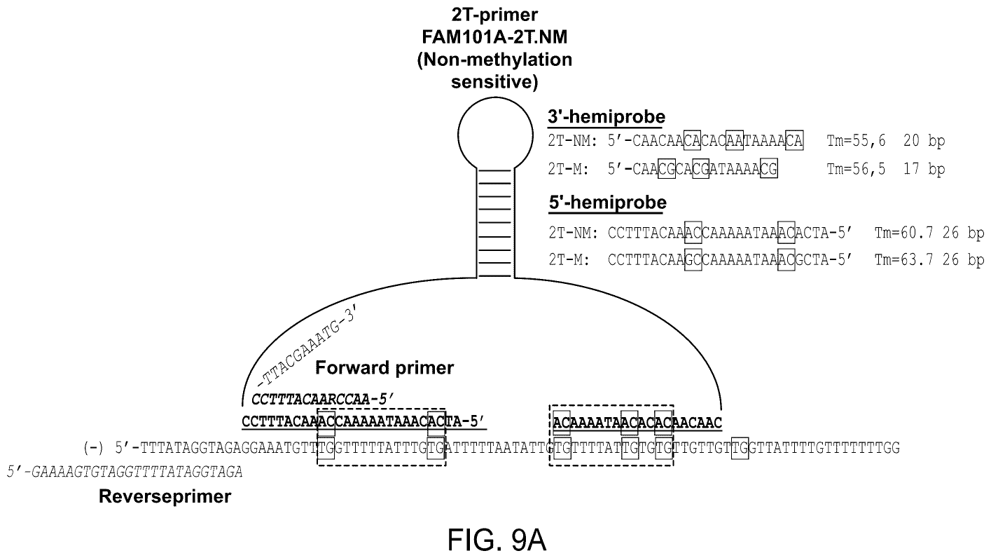

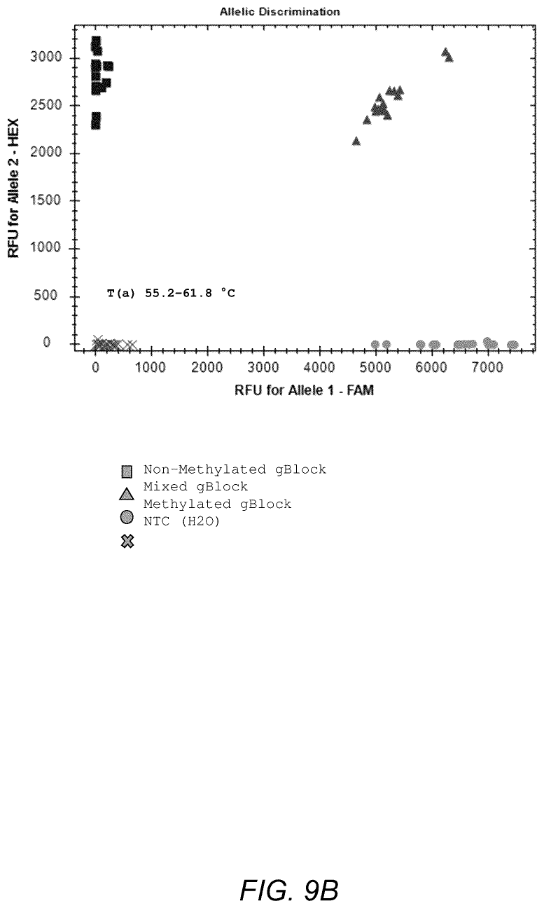

FIG. 9 panel A shows a schematic of a non-methylation-detecting 2T-primer for detecting methylation status of the FAM101A gene (FAM101A-2T.NM with hemiprobes in black text (bold/underlined) and stem loop sequence and arms in dark grey lines, the target sequence in black text, the reverse and forward primer sequence (in black italics). The probe (not shown) binds selectively to the complement of the stem loop sequence and arms of the 2T-primer. The target DNA has been modified so that original “single” cytosine-sites are represented by thymine (since bisulfite-treatment can turn such cytosines into uracils) while original CpG-sites have grey highlight (which in the non-methylated DNA template and in the figure are be represented by TG).

The 3′ hemiprobe of the 2T-primers was designed to interrogate three CpG-sites while the 5′ end interrogate two CpG-sites of FAM101A. Due to the design of the 2T-assay, the length of the interrogated DNA sequence can be kept short, which is an advantage when working with highly fragmented DNA material, such as cfDNA and bisulfite treated DNA. Furthermore, the relatively short 3′ hemiprobe (20 bp) provide excellent discrimination between methylated and non-methylated sequence, in particular as it spans three CpG-sites. In this assay design, also the 5′ hemiprobe increases specificity of the assay as the cooperative binding strength of the 2T-assay will be weaker if not all CpG sites are methylated/un-methylated at the same time.

### Reagents/Procedure

An experiment was performed to evaluate the designed FAM101A-assay, the result of which is shown in FIG. 9 panel B. The template used in the experiment comprising synthetic templates (gBlocks™, IDT), one representing the methylated FAM101A sequence (Methylated gBlock, circles in figure) and one containing the non-methylated FAM101A sequence (Non-methylated gBlock, squares in figure). The template DNA was ordered in such a way that all cytosines appearing alone (not in a CpG dinucleotide) in the sequence was converted into thymine to represent uracils formed after bisulfite-treatment. Cytosines occurring in CpG-dinucleotides were left unchanged in the sequence representing methylated gBlock, while they were changed into thymines in the sequence representing non-methylated gBlock. The two templates were mixed at a 50/50 ratio (Mixed gBlock, triangles in figure) to simulate a tissue with mixed methylation pattern, such as heart tissue (see Ren et al.). A no-template control was also included (NTC (H2O), crosses in figure). The template amount was 2E5 copies/reaction per target sequence.

The reaction volume was 10 μl, and contained TATAA Probe GrandMaster Mix (1×), 400 nM forward and reverse primer, 25 nM FAM101A.2T-M primer (methylation detecting), 25 nM FAM101A.2T-NM primer (non-methylation detecting), 200 nM HEX probe (binding to complement of 2T-M primer), 200 nM FAM probe (binding to complement of 2T-NM primer).

The qPCR reactions were cycled on a BioRad CFX384 with the following thermocycling program: 1 minute at 95° C., 45 cycles each comprising a 5 second denaturation at 95° C. followed by a 55.2-61.8° C. annealing/extension for 30 seconds. Image acquisition was performed in the FAM and HEX channel. The data was analyzed with the CFX Maestro Software using automatic thresholding (single threshold) and baseline adjustment (baseline subtracted curve fit).

The oligonucleotide sequences (5′→3′) used in the experiment are listed in the table below, sequences complementary to probe binding sites are shown for 2T-primer in underlined text:

| Oligo | Sequence |
|---|---|
| FAM101A.2T-M | ATCGCAAATAAAAACCGA |
| | ACATTTCCTTACGAAATC |
| | CAGGTACAGTTGGTACCT |
| | GTCTCCACCCAACGCACG |
| | ATAAAACG |
| FAM101A.2T- | ATCACAAATAAAAACCAAA |
| NM | CATTTCCTTACGAAATCTA |
| | CTAGCGGCAAGCTAGTGCT |
| | AGACTTCAACAACACACAA |
| | TAAAACA |
| 2T-Universal | TACTAGCGGCAAGCTAGTG |
| HEX (non- | CTAGACTT |
| methylation) | |
| probe | |
| 2T-Universal | CAGGTACAGTTGGTACCTG |
| FAM | TCTCCACC |
| (methylation) | |
| probe | |
| FAM101A.F-M | CCGAACATTTCCTTACGAA |
| | ATC |
| FAM101A.F-NM | AACCAAACATTTCCTTACG |
| | AAATC |
| FAM101A.R | GAAAAGTGTAGGTTTTATA |
| | GGTAGA |

### Results

FIG. 9 panel B shows allelic discrimination performance of the 2T-assay in qPCR on synthetic gBlock sequences representing methylated DNA, non-methylated DNA, mixed methylated/non-methylated DNA and a no template control (NTC). At annealing temperatures of around 55-62° C., the assay performs robustly and a clear distinction of different type of template DNA is made while keeping false-positive signal very low and limited to the FAM-channel (detecting methylated DNA).

### Example 10.—Detection of Tissue Specific DNA by Targeting a Selective Methylation Pattern

CG-islands in CORO6 and FAM101A have been documented to have high and low degree of methylation in heart tissue, respectively, opposing the patterns observed in other tissues, such as white blood cells (see e.g. Ren et al. 2022, Zemmour et al. 2018, which is incorporated by reference in its entirety herein). Having demonstrated the effectiveness of the 2-tailed approach of FIG. 1 for CORO6 and FAM101A in the previous examples, we hypothesized distinguishing heart DNA in cell free DNA present in plasma may be possible.

### Reagents/Procedure

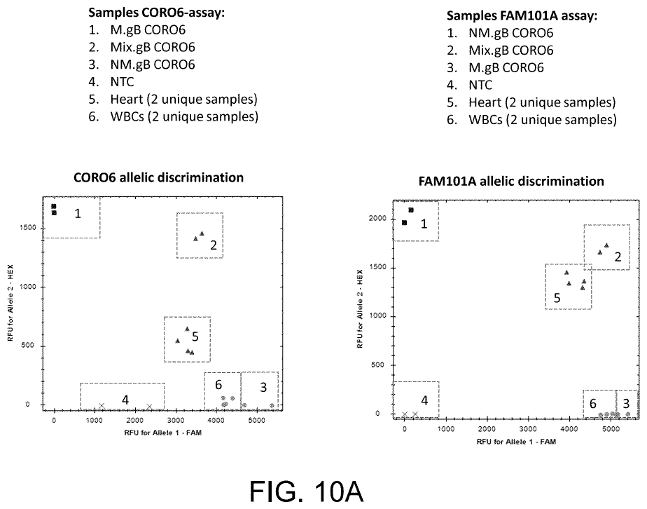

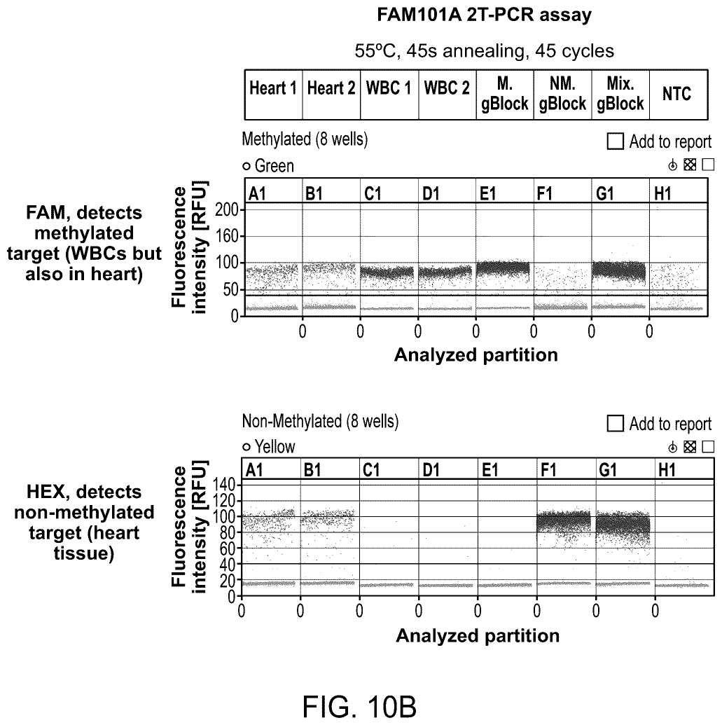

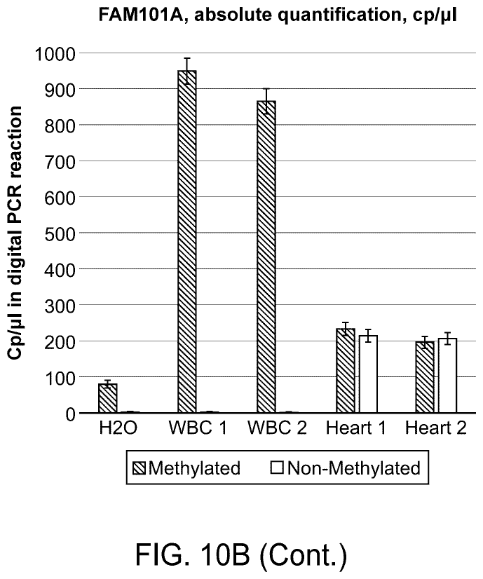

In order to evaluate the tissue discriminatory capacity of the assays, the assays were used to analyze DNA extracted from heart tissue and white blood cells (WBCs). The diagram in FIG. 10 Panel A represent allelic discrimination plots based on the final relative fluorescence (RFU) obtained in a qPCR experiment in which the 2T-PCR assays CORO6 and FAM101A described previously were used to discriminate between methylated and non-methylated DNA.

The synthetic templates used were the same listed in method section for FIGS. 8 and 9: representing methylated CORO6/FAM101A sequence (M.gBlock) and non-methylated CORO6/FAM101A sequence (NM. gBlock). The templates were mixed at a 50/50 ratio (Mixed gBlock) to simulate a tissue with mixed methylation pattern, such as heart tissue (Ren et al. 2022, which is incorporated by reference in its entirety herein). The template amount was 2E5 copies/reaction per target sequence. A no-template control was also included (NTC).

The human derived samples came from two unique WBC samples collected from pooled blood samples in EDTA-tubes from 20-30 individuals, and two heart samples obtained from two unique patients undergoing heart surgery. The DNA was extracted from the cells/tissues using a DNeasy Blood & Tissue Kit (Qiagen, art no 69504). 200 ng of the each extracted DNA sample were then bisulfite-treated (BST) using an EZ DNA Methylation-Lightning Kit (Zymo Research, art no. D5030) and eluted in 10 μl elution buffer. 2 μl of the eluate was used as template for the subsequent qPCR-analysis, corresponding to 40 ng of BST-DNA. The bisulfite-treatment is expected to degrade DNA to a level of 60-90% (Zemmour et al. 2018).

The reaction volume was 10 μl, and contained TATAA Probe GrandMaster Mix (1×), 400 nM forward and reverse primer, 25 nM CORO6/FAM101A.2T-M primer (methylation detecting), 25 nM CORO6/FAM101A.2T-NM primer (non-methylation detecting), 200 nM HEX probe (binding to complement of 2T-M (CORO6) or 2T-NM (FAM101A) primer), 200 nM FAM probe (binding to complement of 2T-NM (CORO6) or 2T-M (FAM101A) primer). The oligonucleotide sequences used in the experiment were the same as listed in reagents/procedure section in example 8 and 9.

The qPCR reactions were cycled on a BioRad CFX384 with the following thermocycling program: 1 minute at 95° C., 45 cycles each comprising a 5 second denaturation at 95° C. followed by a 60° C. annealing/extension for 30 seconds. Image acquisition was performed using the FAM and HEX channel. The data was analyzed with the CFX Maestro Software using automatic thresholding (single threshold) and baseline adjustment (baseline subtracted curve fit).

The samples described in method above for FIG. 10 Panel A were also analyzed in a digital PCR experiment using the FAM101A assay oligonucleotide described in reagents/procedure for example 9. Each dPCR reaction was 12 μl and contained QIAcuity Probe Mastermix (1×), 800 nM forward and reverse primer, 25 nM FAM101A.2T-M primer (methylation detecting), 25 nM FAM101A.2T-NM primer (non-methylation detecting), 200 nM HEX probe (binding to complement of 2T-NM primer), 200 nM FAM probe (binding to complement of 2T-M primer). 4 μl template was added to each reaction. For the synthetic gBlock samples, the stock had a concentration of 1E4 cp/μl (total loading per reaction: 40.000 copies per target), while the tissue DNA had concentration of 20 ng/μl before BST-treatment (total loading per reaction, 80 ng, corresponding to approximately 24.000 genome copies). The oligonucleotide sequences used in the experiment were the same as listed in table 1 and 2.

The dPCR reactions were loaded into a QIAcuity Nanoplate 26K and cycled on the QIAcuity Digital PCR System with the following conditions: 3 minutes at 95° C., 45 cycles each comprising a 15 second denaturation at 95° C. followed by a 55° C. extension for 30 seconds. Image acquisition was performed in the FAM and HEX channel. The data was analyzed with the QIAcuity Software Suite using manual thresholding.

### Results

FIG. 10 panel A depicts qPCR allelic discrimination results when analysing of methylation/non-methylation representative gBlocks of genes CORO6 and FAM101A synthetically produced (2E5 cp/reaction) (M.gB—methylated target; NM.gB—non-methylated target; Mix.gB—50/50 mix of M/NM-gBlocks) alongside two unique bisulfite treated (BST) DNA samples extracted from white blood cells (WBCs) and heart tissue (40 ng DNA/reaction (before BST)). CORO6 and FAM101A primers were constructed as in previous examples, and qPCR to detect both markers was performed as in previous examples. HEX fluorophore is a signal for methylated CORO6 sequence and non-methylated FAM101A. As predicted by the documented behaviour of CORO6 and FAM101A, WBCs show signal in the FAM-channel, while heart show signal in both HEX- and FAM-channel, showing that both assays can detect heart DNA in a background of white blood cells (the main source of DNA in cfDNA).

FIG. 10 panel B depicts results when samples described in FIG. 10 panel A were analysed with the FAM101A 2T-assay using digital PCR (QIAcuity, Qiagen) instead of qPCR. Heart samples show a mixed signal (FAM/HEX), while WBCs show signal for the methylated DNA (FAM). The NTC show a relatively high background fluorescence in the NTC, but the signal is limited to the FAM-channel, and as such detection of heart-specific signal (HEX) is not compromised. These results indicate 2T-PCR will be effective for quantifying total target DNA in the context of methylation (non-methylated/methylated), which be beneficial for analysis of cfDNA, where analysis of tissue of origin for the cfDNA is biologically informative.

### Example 11.—Direct Blood Genotyping

Having observed high performance of the two-tailed method of FIG. 1 for detecting mutations in a variety of contexts, we asked whether the method can be effective for genotyping from crude blood samples. Accordingly, a crude blood sample, (e.g. without extraction or purification of the nucleic acid) was genotyped for factor V (“Leiden”) mutation using 2T-qPCR. Primers were designed according to the scheme depicted in FIG. 1 and the previous examples to generate SEQ ID NOs: 67-72. Three standard blood samples (homozygote wild type, homozygote mutant and heterozygote) were obtained from Equalis. 1 μl of each blood sample were incubated with 24 ul lysis buffer at 96 degrees C. for 10 minutes. The sample was centrifuged to remove cell debris. The supernatant was then mixed with equal amount of water and 2 μl was used at template in a qPCR using conditions described in the previous examples.

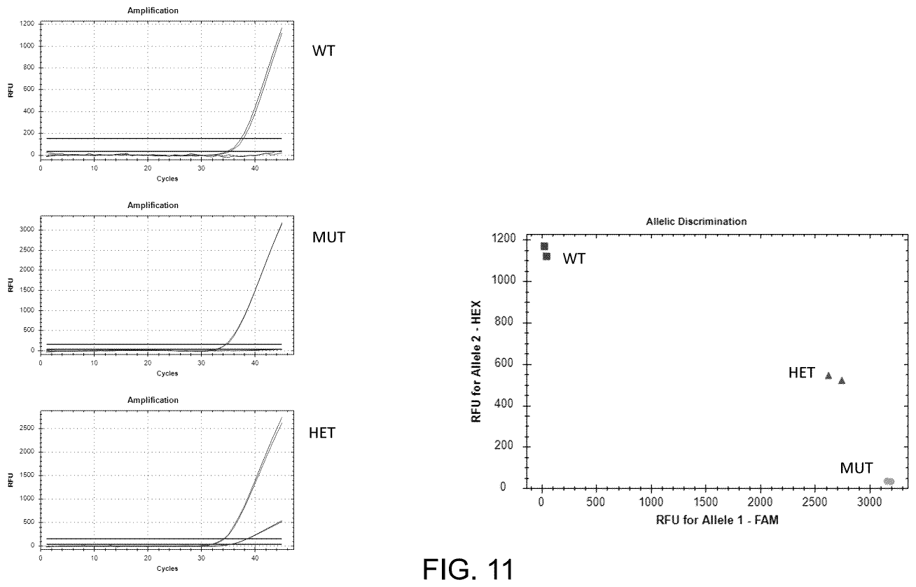

FIG. 11 depicts results of this experiment. The left panel of FIG. 11 shows duplicate qPCR measurement on homozygote wild type (top), homozygote mutant (middle) and heterozygote (bottom). The right panel shows a plot clustering the measured data based on fluorescence intensity clearly distinguishing the duplicate two homoduplexes and the heteroduplex. The plot in the right panel clearly demonstrates that wild-type, heterozygous, and mutant can be discriminated from whole crude blood without additional purification steps.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

1. A method for processing a DNA sequence having or suspected of having sequence variation relative to a wild-type sequence, said method comprising:
combining in a reaction mixture suitable for processing said DNA sequence:
(i) said DNA sequence, wherein said DNA sequence comprises a variation of at least one nucleotide relative to said wild-type sequence;
(ii) a stem-loop primer that comprises:
a 5′ hemiprobe sequence configured to hybridize to a complementary first end region of said DNA sequence;
a stem-loop sequence; and
a 3′ hemiprobe sequence configured to hybridize to a second end region of said DNA sequence,
wherein a 3′ portion of said 3′ hemiprobe sequence comprises a nucleotide sequence complementary to said variation of said at least one nucleotide relative to said wild-type sequence but not complementary to said wild-type sequence; and
(iii) a reverse primer configured to hybridize to a genomic region 3′ from said variation of said at least one nucleotide relative to said wild-type sequence; and
incubating said reaction mixture under conditions suitable to extend a product containing said 3′ hemiprobe sequence.

2. (canceled)

3. (canceled)

4. (canceled)

5. The method of claim 1, further comprising incubating said reaction mixture under conditions suitable to produce extension products from said reverse primer.

6. The method of claim 5, further comprising combining in said reaction mixture or a second reaction mixture said extension products produced from said reverse primer and a forward primer configured to hybridize to a region of said extension products complementary to: (i) a portion of said 5′ hemiprobe sequence; and (ii) a portion of a stem of said stem-loop sequence.

7. The method of claim 6, wherein said forward primer comprises at least 12 nucleotides, and comprises at least 7 nucleotides complementary to a complement of said portion of said 5′ hemiprobe sequence.

8. The method of claim 7, wherein said forward primer comprises at least 9 nucleotides complementary to a complement of said portion of said stem of said stem-loop sequence.

9. (canceled)

10. The method of claim 6, further comprising incubating said second reaction mixture or said reaction mixture containing: (a) said extension products produced from said reverse primer; and (b) said forward primer under conditions suitable to produce extension products from said forward primer.

11. The method of claim 6, wherein the concentrations of said reverse primer and said forward primer in said reaction mixture or said second reaction mixture are in excess of the concentration of said stem-loop primer.

12. The method of claim 6, wherein the concentration of said stem-loop primer in said reaction mixture or said second reaction mixture is in excess of the concentration of said DNA sequence.

13. (canceled)

14. The method of claim 1, wherein said DNA sequence comprises genomic DNA.

15. The method of claim 1, wherein said 5′ hemiprobe sequence is at least 7 nucleotides in length.

16. The method of claim 1, wherein said 3′ hemiprobe sequence is at least 3 nucleotides in length.

17. (canceled)

18. The method of claim 1, wherein said stem-loop sequence comprises 15 nucleotides.

19. The method of claim 1, wherein said stem-loop sequence is configured to have a Tm of about 55 to about 75 degrees Celsius.

20. The method of claim 1, wherein a loop of said stem-loop sequence is 1 nucleotide to 20 nucleotides in length.

21. The method of claim 1, wherein a loop of said stem-loop sequence comprises a barcode.

22. The method of claim 10, wherein said reaction mixture or said second reaction mixture further comprises an oligonucleotide probe comprising a detectable moiety, wherein said oligonucleotide probe is configured to hybridize to a complement of at least part of said stem-loop primer.

23. The method of claim 22, wherein said at least part of said stem-loop primer comprises at least part of said stem-loop sequence.

24. The method of claim 23, wherein said at least part of said stem-loop sequence comprises at least part of a loop sequence within said stem-loop sequence.

25. The method of claim 22, wherein said detectable moiety comprises a 5′ fluorophore.

26. The method of claim 25, wherein said oligonucleotide probe comprising said detectable moiety further comprises a quencher.

27. The method of claim 10, further comprising performing a PCR reaction, a qPCR reaction, a dPCR reaction, a ddPCR reaction, or a sequencing reaction.

28. A kit for processing a DNA sequence having or suspected of having sequence variation relative to a wild-type sequence, said kit comprising:
(a) a stem-loop primer that comprises:
(i) a 5′ hemiprobe sequence configured to hybridize to a complementary first end region of said DNA sequence;
(ii) a stem-loop sequence; and
(iii) a 3′ hemiprobe sequence configured to hybridize to a second end region of said DNA sequence;
(b) a forward primer configured to hybridize to a region complementary to: (i) at least part of said 5′ hemiprobe sequence; and (ii) at least part of a stem of said stem-loop sequence; and
(c) a reverse primer configured to hybridize to a genomic region of said DNA sequence that is 3′ from said sequence variation.

29-48. (canceled)

49. A composition for processing a DNA sequence having or suspected of having sequence variation relative to a wild-type sequence, said composition comprising:
(a) a stem-loop primer that comprises:
(i) a 5′ hemiprobe sequence configured to hybridize to a complementary first end region of said DNA sequence;
(ii) a stem-loop sequence; and
(iii) a 3′ hemiprobe sequence configured to hybridize to a second end region of said DNA sequence;
(b) a forward primer configured to hybridize to a region complementary to: (i) at least part of said 5′ hemiprobe sequence; and (ii) at least part of a stem of said stem-loop sequence; and
(c) a reverse primer configured to hybridize to a genomic region 3′ from said sequence variation, wherein
the concentration of said forward primer or the concentration of said reverse primer in said composition is at least 10-fold higher than the concentration of said stem-loop primer.

50-71. (canceled)

72. A method for processing a DNA sequence having or suspected of having a methylated cytosine, said method comprising:
combining in a reaction mixture suitable for processing said DNA sequence:
(i) said DNA sequence, wherein said DNA sequence comprises a uridine residue; and
(ii) a first stem-loop primer that comprises:
a 5′ hemiprobe sequence configured to hybridize to a complementary first end region of said DNA sequence;
a stem-loop sequence; and
a 3′ hemiprobe sequence configured to hybridize to a second end region of said DNA sequence, wherein a 3′ portion of said 3′ hemiprobe sequence comprises a nucleotide complementary to said uridine residue but not complementary to a cytidine residue.

73-84. (canceled)
